(19) United States
(12) Patent Application Publication
(10) Pub. No.: US 2025/0369123 A1
(43) Pub. Date: Dec. 4, 2025

(54) ALKOXYIMIDAZOLINIUM QUATERNARY SALT COMPOSITIONS AND METHODS OF USING THE COMPOSITIONS TO INHIBIT CORROSION

(71) Applicant: Ecolab USA Inc., St. Paul, MN (US)

(72) Inventors: Jonathan MASERE, Richmond, TX (US); Ashish DHAWAN, Aurora, IL (US); Kameswara VYAKARANAM, Sugar Land, TX (US)

(73) Assignee: Ecolab USA Inc., St. Paul, MN (US)

(21) Appl. No.: 19/220,939

(22) Filed: May 28, 2025

Related U.S. Application Data
(60) Provisional application No. 63/654,918, filed on May 31, 2024.

(51) Int. Cl. C23F 11/14; C07D 233/14

(52) U.S. Cl. CPC C23F 11/149; C07D 233/14

(57) ABSTRACT

Corrosion inhibitor compositions that include a compound of formula (I) are provided. In general, the compound of formula (I) is an environmentally benign quaternarized imidazoline having a lipophilic group. A method of reducing corrosion of a metal surface in contact with an aqueous system that includes adding the disclosed corrosion inhibitor composition to the aqueous system is also provided. Also provided are processes for preparing corrosion inhibitor compounds of formula (I).

### TECHNICAL FIELD

The present disclosure generally relates to corrosion inhibitor compositions and methods of inhibiting corrosion using the compositions. More particularly, the disclosure pertains to compositions including alkoxyimidazolinium quaternary salts and methods of inhibiting corrosion using the same.

### BACKGROUND

Equipment and infrastructure used in the recovery, transportation storage, and processing of oil and natural gas come into contact with highly corrosive agents. These corrosive agents include brines, organic acids, carbon dioxide and hydrogen sulfide. In addition to exposure to these corrosive agents, damage to the equipment and infrastructure is frequently exacerbated by erosion from insoluble materials including sand and other inorganic solids.

Processing equipment is particularly vulnerable to damage from corrosive agents given the metallurgical properties of the materials used to fabricate this equipment. Common metals used in processing equipment that are particularly vulnerable to damage from corrosion and erosion include metallic chrome steel compositions, ferritic alloy steel compositions, austenitic steel compositions, precipitation-hardened steel compositions, and high nickel steel compositions. In particular, carbon steel compositions are frequently used in the fabrication of processing equipment, given its comparatively low cost.

The high vulnerability of the materials used to fabricate oil and gas equipment to corrosion makes the use of corrosion inhibitors a necessity. Unfortunately, a major constraint in this field is the limited selection of environmentally benign corrosion inhibitors. Waste streams containing these corrosion inhibitors are liable to end up in the environment, for example, through accidental leakage or routine disposal. Of the most commonly used corrosion inhibitors, few are derived from sustainable sources and the vast majority are not environmentally benign. Consequently, there is a significant unmet need for environmentally benign corrosion inhibitors for protecting equipment and infrastructure used in the production, transportation, storage, and processing of oil and gas. The discovery and implementation of such corrosion inhibitors is essential for maintaining equipment and infrastructure integrity with minimal damage to the environment.

To be effective, corrosion inhibitors, must typically have long lipophilic hydrocarbon moieties and compact polar functional head groups at one end of the lipophilic chain. Without being bound by theory, corrosion inhibitors of this type function as surface-active compounds, where the polar head groups bind to the metal surface while the non-polar tails protrude away from the metal surface. The resultant hydrophobic films formed by the hydrophobic tails protruding from the metal surface protect the metal surfaces from water-borne corrosive agents.

One method for improving the effectiveness of corrosion inhibitors is to increase the overall polarity of the polar head groups. Amphiphilic quaternary ammonium compounds have been developed and employed as effective corrosion inhibitors, but many are not derived from sustainable sources and are, likewise, not biodegradable or environmentally benign. Thus, there is a significant need to develop environmentally benign and biodegradable corrosion inhibitors that are derived from sustainable sources.

Subjected to harsh chemical environments, hydroxyethyl imidazolines tend to be unstable. In acidic media, for example, the protonation of the 1 N position of the imidazoline ring tends to lead to ring-opening hydrolysis. Corrosion inhibitors used in the oil and gas industry are frequently subjected to acidic aqueous media, limiting the utility of acid-unstable corrosion inhibitors like the hydroxyethyl imidazolines.

### BRIEF SUMMARY

Provided herein are corrosion inhibitor compositions that include a compound of formula (I)

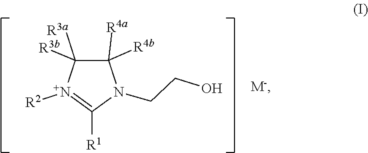

or a salt thereof, wherein:
R¹ is an optionally substituted C₈-C₂₀ alkyl, an optionally substituted C₈-C₂₀ alkenyl with one to multiple vinylic moieties, or an optionally substituted C₈-C₂₀ alkynyl with one to multiple vinylic moieties, an optionally substituted —(C₆-C₂₀ aryl)-(C₄-C₂₀ alkyl), an optionally substituted
—(C₄-C₂₀ alkylene with one to multiple vinylic moieties)-(C₆-C₂₀ aryl), or an optionally substituted
—(C₄-C₂₀ alkylene with one to multiple vinylic moieties)-(C₆-C₂₀ aryl)-(C₄-C₂₀ alkyl);
R² is optionally substituted C₁-C₂₀ alkyl, optionally substituted
C₂-C₂₀ hydroxyalkyl, optionally substituted —(C₁-C₂₀ alkylene)-(C₆-C₂₀ aryl), optionally substituted —(C₂-C₂₀ hydroxyalkylene)-(C₆-C₂₀ aryl), or optionally substituted —(C₂-C₂₀ hydroxyalkylene)-(C₆-C₂₀ aryl)-(C₆-C₂₀ alkyl);
R^{3a} is H, —OH, —NH₂, —NH(C₁-C₆ alkyl), —N(C₁-C₆ alkyl)₂, —CN, halo, or optionally substituted C₁-C₆ alkyl;
R^{3b} is H, —OH, —NH₂, —NH(C₁-C₆ alkyl), —N(C₁-C₆ alkyl)₂, —CN, halo, or optionally substituted C₁-C₆ alkyl;
R^{4a} is H, —OH, —NH₂, —NH(C₁-C₆ alkyl), —N(C₁-C₆ alkyl)₂, —CN, halo, or optionally substituted C₁-C₆ alkyl;
R^{4b} is H, —OH, —NH₂, —NH(C₁-C₆ alkyl), —N(C₁-C₆ alkyl)₂, —CN, halo, or optionally substituted C₁-C₆ alkyl; and
M⁻ is a counter ion.

In some embodiments, the corrosion inhibitor composition includes a compound of formula (I-A):

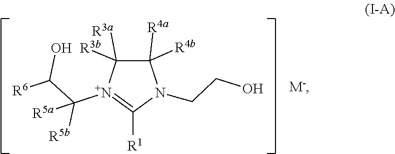

wherein:
R¹ is an optionally substituted C₈-C₂₀ alkyl, an optionally substituted
C₈-C₂₀ alkeny with one to multiple vinylic moieties I, or an optionally substituted C₈-C₂₀ alkynyl with one to multiple vinylic moieties;
R^{3a} is H, —OH, —NH₂, —NH(C₁-C₆ alkyl), —N(C₁-C₆ alkyl)₂, —CN, halo, or optionally substituted C₁-C₆ alkyl;
R^{3b} is H, —OH, —NH₂, —NH(C₁-C₆ alkyl), —N(C₁-C₆ alkyl)₂, —CN, halo, or optionally substituted C₁-C₆ alkyl;
R^{4a} is H, —OH, —NH₂, —NH(C₁-C₆ alkyl), —N(C₁-C₆ alkyl)₂, —CN, halo, or optionally substituted C₁-C₆ alkyl;
R^{4b} is H, —OH, —NH₂, —NH(C₁-C₆ alkyl), —N(C₁-C₆ alkyl)₂, —CN, halo, or optionally substituted C₁-C₆ alkyl;
R^{5a} is H, —OH, —NH₂, —NH(C₁-C₆ alkyl), —N(C₁-C₆ alkyl)₂, —CN, halo, or optionally substituted C₁-C₆ alkyl;
R^{5b} is H, —OH, —NH₂, —NH(C₁-C₆ alkyl), —N(C₁-C₆ alkyl)₂, —CN, halo, or optionally substituted C₁-C₆ alkyl;
R⁶ is optionally H, substituted C₁-C₂₀ alkyl, optionally substituted
C₁-C₂₀ aryl, or optionally substituted —(C₆-C₂₀ aryl)-(C₄-C₂₀ alkyl); and
M⁻ is a counter ion.

A method of reducing corrosion of a metal surface in contact with an aqueous system is also provided, where the method includes adding a corrosion inhibitor composition of a compound of formula (I) to the aqueous system. The aqueous system may include one or more corrosive agents, including, but not limited to, brine, organic acids, carbon dioxide, and hydrogen sulfide. The metal surface of the aqueous system may include metallic-chrome steel, ferritic-alloy steel, austenitic-steel, precipitation-hardened steel, high-nickel steel, carbon steel, or a combination thereof.

Also provided is a process for preparing a compound of formula (I-A) that includes contacting a compound of formula (I-B):

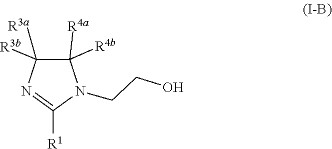

with a compound of formula (I-C):

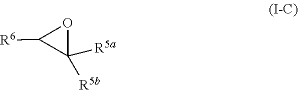

wherein:
R¹ is optionally substituted C₈-C₂₀ alkyl, an optionally substituted
C₈-C₂₀ alkenyl with one to multiple vinylic moieties, or an optionally substituted C₈-C₂₀ alkynyl with one to multiple vinylic moieties, an optionally substituted —(C₆-C₂₀ aryl)-(C₄-C₂₀ alkyl), an optionally substituted
—(C₄-C₂₀ alkylene)-(C₆-C₂₀ aryl), or an optionally substituted
—(C₄-C₂₀ alkylene)-(C₆-C₂₀ aryl)-(C₄-C₂₀ alkyl);
R^{3a} cis H, —OH, —NH₂, —NH(C₁-C₆ alkyl), —N(C₁-C₆ alkyl)₂, —CN, halo, or optionally substituted C₁-C₆ alkyl;
R^{3b} is H, —OH, —NH₂, —NH(C₁-C₆ alkyl), —N(C₁-C₆ alkyl)₂, —CN, halo, or optionally substituted C₁-C₆ alkyl;
R^{4a} is H, —OH, —NH₂, —NH(C₁-C₆ alkyl), —N(C₁-C₆ alkyl)₂, —CN, halo, or optionally substituted C₁-C₆ alkyl;
R^{4b} is H, —OH, —NH₂, —NH(C₁-C₆ alkyl), —N(C₁-C₆ alkyl)₂, —CN, halo, or optionally substituted C₁-C₆ alkyl; and
R^{5a} is H, —OH, —NH₂, —NH(C₁-C₆ alkyl), —N(C₁-C₆ alkyl)₂, —CN, halo, or optionally substituted C₁-C₆ alkyl;
R^{5b} is H, —OH, —NH₂, —NH(C₁-C₆ alkyl), —N(C₁-C₆ alkyl)₂, —CN, halo, or optionally substituted C₁-C₆ alkyl;
R⁶ is optionally substituted C₁-C₆ alkyl or optionally substituted C₆-C₁₀ aryl; and
M⁻ is a counter ion.

The foregoing has outlined rather broadly the features and technical advantages of the present disclosure in order that the detailed description that follows may be better understood. Additional features and advantages of the disclosure will be described hereinafter that form the subject of the claims of this application.

### DETAILED DESCRIPTION

Various embodiments are described below. The relationship and functioning of the various elements of the embodiments may better be understood by reference to the following detailed description. However, embodiments are not strictly limited to those described below.

Examples of methods and materials are described below, although methods and materials similar or equivalent to those described herein can be used in practice or testing of the present disclosure. All publications, patent applications, patents and other reference materials mentioned herein are incorporated by reference in their entirety. The materials, methods, and examples disclosed herein are illustrative only and not intended to be limiting.

Unless otherwise defined, all technical and scientific terms used herein have the same meaning as commonly understood by one of ordinary skill in the art. In case of conflict, the present document, including definitions, will control.

Unless otherwise indicated, an alkyl group as described herein alone or as part of another group is an optionally substituted linear or branched saturated monovalent hydrocarbon substituent containing from, for example, one to about sixty carbon atoms, such as one to about thirty carbon atoms, in the main chain. Examples of unsubstituted alkyl groups include methyl, ethyl, n-propyl, i-propyl, n-butyl, i-butyl, s-butyl, t-butyl, n-pentyl, i-pentyl, s-pentyl, t-pentyl, and the like.

Any alkyl functional group disclosed herein may be derived from, for example, an animal fatty acid or a vegetable oil fatty acid, such as a soy fatty acid, a tall oil fatty acid, a canola oil fatty acid, oleic acid, glycerol-restricted avocado oil fatty acid, corn oil fatty acid, cottonseed oil fatty acid, grape seed oil fatty acid, hazelnut oil fatty acid, hemp seed oil fatty acid, linseed oil fatty acid, olive oil fatty acid, palm kernel oil fatty acid, peanut seed oil fatty acid, rape seed oil fatty acid, rice bran oil fatty acid, safflower oil fatty acid, sesame oil fatty acid, soybean oil fatty acid, sunflower seed oil fatty acid, and walnut oil fatty acid. In some instances, a glycerol-restricted vegetable oil fatty acid may comprise, consist of, or consist essentially of glycerol-restricted soybean oil fatty acid.

“Alkenyl” refers to a straight or branched hydrocarbon having, for example, 2, 3, 4, 5, 6, 7, 8, 9, 10, 11, 12, 13, 14, 15, or 16 carbon atoms, and having one or more carbon-carbon double bonds. Alkenyl groups include, but are not limited to, ethenyl, 1-propenyl, 2-propenyl (allyl), iso-propenyl, 2-methyl-1-propenyl, 1-butenyl, and 2-butenyl. Alkenyl groups may be unsubstituted or substituted by one or more suitable substituents.

“Alkynyl” refers to a straight or branched hydrocarbon having, for example, 2, 3, 4, 5, 6, 7, 8, 9, 10, 11, 12, 13, 14, 15, or 16 carbon atoms, and having one or more carbon-carbon triple bonds. Alkynyl groups include, but are not limited to, ethynyl, propynyl, and butynyl. Alkynyl groups may be unsubstituted or substituted by one or more suitable substituents.

“Halogen” or “halo” refers to F, Cl, Br, and I.

The terms “aryl” or “ar” as used herein alone or as part of another group (e.g., arylene) denote optionally substituted homocyclic aromatic groups, such as monocyclic or bicyclic groups containing from about 6 to about 12 carbons in the ring portion, such as phenyl, biphenyl, naphthyl, substituted phenyl, substituted biphenyl or substituted naphthyl. The term “aryl” also includes heteroaryl functional groups. It is understood that the term “aryl” applies to cyclic substituents that are planar and comprise 4n+2 electrons, according to Huckel's Rule.

“Heteroaryl” refers to a monocyclic or bicyclic 5- or 6-membered ring system, wherein the heteroaryl group is unsaturated and satisfies Huckel's rule. Non-limiting examples of heteroaryl groups include furanyl, thiophenyl, pyrrolyl, pyrazolyl, imidazolyl, 1,2,3-triazolyl, 1,2,4-triazolyl, isoxazolyl, oxazolyl, isothiazolyl, thiazolyl, 1,3,4-oxadiazol-2-yl, 1,2,4-oxadiazol-2-yl, 5-methyl-1,3,4-oxadiazole, 3-methyl-1,2,4-oxadiazole, pyridinyl, pyrimidinyl, pyrazinyl, triazinyl, benzofuranyl, benzothiophenyl, indolyl, quinolinyl, isoquinolinyl, benzimidazolyl, benzoxazolinyl, benzothiazolinyl, quinazolinyl, and the like.

Compounds of the present disclosure may be substituted with suitable substituents. The term “suitable substituent,” as used herein, is intended to mean a chemically acceptable functional group, preferably a moiety that does not negate the activity of the compounds. Such suitable substituents include, but are not limited to, halo groups, perfluoroalkyl groups, perfluoro-alkoxy groups, alkyl groups, alkenyl groups, alkynyl groups, hydroxy groups, oxo groups, mercapto groups, alkylthio groups, alkoxy groups, aryl or heteroaryl groups, aryloxy or heteroaryloxy groups, aralkyl or heteroaralkyl groups, aralkoxy or heteroaralkoxy groups, HO—(C═O)— groups, heterocylic groups, cycloalkyl groups, amino groups, alkyl- and dialkylamino groups, carbamoyl groups, alkylcarbonyl groups, alkoxycarbonyl groups, alkylaminocarbonyl groups, dialkylamino carbonyl groups, arylcarbonyl groups, aryloxy-carbonyl groups, alkylsulfonyl groups, and arylsulfonyl groups. In some embodiments, suitable substituents may include halogen, an unsubstituted C₁-C₁₂ alkyl group, an unsubstituted C₄-C₆ aryl group, or an unsubstituted C₁-C₁₀ alkoxy group. Those skilled in the art will appreciate that many substituents can be substituted by additional substituents.

The term “substituted” as in “substituted alkyl,” means that in the group in question (i.e., the alkyl group), at least one hydrogen atom bound to a carbon atom is replaced with one or more substituent groups, such as hydroxy (—OH), alkylthio, phosphino, amido (—CON(R_{A})(R_{B}), wherein R_{A} and R_{B} are independently hydrogen, alkyl, or aryl), amino(—N(R_{A})(R_{B}), wherein R_{A} and R_{B} are independently hydrogen, alkyl, or aryl), halo (fluoro, chloro, bromo, or iodo), silyl, nitro (—NO₂), an ether (—OR_{A} wherein R_{A} is alkyl or aryl), an ester (—OC(O)R_{A} wherein R_{A} is alkyl or aryl), keto (—C(O)R_{A} wherein R_{A} is alkyl or aryl), heterocyclo, and the like.

When the term “substituted” introduces a list of possible substituted groups, it is intended that the term apply to every member of that group. That is, the phrase “optionally substituted alkyl or aryl” is to be interpreted as “optionally substituted alkyl or optionally substituted aryl.”

“Aqueous system” refers to any system containing one or more metallic surfaces/components, which are in contact with water on a periodic or continuous basis.

“Industrial water system” means any system that circulates water as a component. Non-limiting examples of “industrial water systems” include cooling systems, boiler systems, heating systems, membrane systems, paper making systems, food and beverage systems, oil and gas systems, and any other system that circulates or includes water.

The present disclosure relates to corrosion inhibitor compositions, methods of inhibiting corrosion, and formulations useful for inhibiting corrosion. Inhibiting corrosion includes, for example, reducing corrosion, completely eliminating corrosion or prohibiting corrosion from occurring for some period of time, lowering a rate of corrosion, etc. In some aspects, the corrosion inhibitor compositions are useful for inhibiting corrosion of metallic surfaces in aqueous environments. In some aspects, the corrosion inhibitor compositions and/or formulations comprise one or more alkoxyimidazolinium quaternary salt having a lipophilic tail group.

The present disclosure addresses the shortfalls of the prior art by describing novel corrosion inhibitor compounds that are alkoxylated quaternary salts of imidazolines derived from renewable fatty acids with surprisingly improved efficacy as corrosion inhibitors. Without being bound by theory, quaternization through alkoxylation of the imidazoline core stabilizes the corrosion inhibitor against acidic media and broadens the potential fields of use for the corrosion inhibitors. Thus, the present disclosure addresses a significant unmet need by providing effective, environmentally benign corrosion inhibitors for use in a variety of applications, including the oil and natural gas industries.

In some embodiments, the present disclosure provides a corrosion inhibitor composition comprising a compound of formula (I):

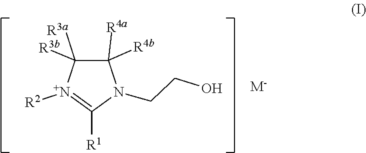

or a salt thereof, wherein:
R¹ is an optionally substituted C₈-C₂₀ alkyl, an optionally substituted
C₈-C₂₀ alkenyl with one to multiple vinylic moieties, an optionally substituted C₈-C₂₀ alkynyl with one to multiple vinylic moieties, an optionally substituted —(C₆-C₂₀ aryl)-(C₄-C₂₀ alkyl), an optionally substituted
—(C₄-C₂₀ alkylene)-(C₆-C₂₀ aryl), or an optionally substituted
—(C₄-C₂₀ alkylene)-(C₆-C₂₀ aryl)-(C₄-C₂₀ alkyl);
R² is optionally substituted C₁-C₂₀ alkyl, optionally substituted
C₂-C₂₀ hydroxyalkyl, optionally substituted —(C₁-C₂₀ alkylene)-(C₆-C₂₀ aryl), optionally substituted —(C₂-C₂₀ hydroxyalkylene)-(C₆-C₂₀ aryl), or optionally substituted —(C₂-C₂₀ hydroxyalkylene)-(C₆-C₂₀ aryl)-(C₆-C₂₀ alkyl);
R^{3a} is H, —OH, —NH₂, —NH(C₁-C₆ alkyl), —N(C₁-C₆ alkyl)₂, —CN, halo, or optionally substituted C₁-C₆ alkyl;
R^{3b} is H, —OH, —NH₂, —NH(C₁-C₆ alkyl), —N(C₁-C₆ alkyl)₂, —CN, halo, or optionally substituted C₁-C₆ alkyl;
R^{4a} is H, —OH, —NH₂, —NH(C₁-C₆ alkyl), —N(C₁-C₆ alkyl)₂, —CN, halo, or optionally substituted C₁-C₆ alkyl;
R^{4b} is H, —OH, —NH₂, —NH(C₁-C₆ alkyl), —N(C₁-C₆ alkyl)₂, —CN, halo, or optionally substituted C₁-C₆ alkyl; and
M⁻ is a counter ion.

In some embodiments, the compound of formula (I) is a compound of formula (I-A):

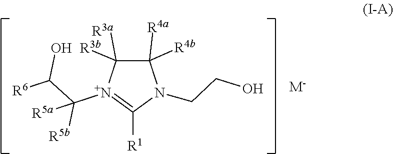

wherein:
R¹ is an optionally substituted C₈-C₂₀ alkyl, an optionally substituted
C₈-C₂₀ alkenyl with one to multiple vinylic moieties, or an optionally substituted C₈-C₂₀ alkynyl with one to multiple vinylic moieties;
R^{3a} is H, —OH, —NH₂, —NH(C₁-C₆ alkyl), —N(C₁-C₆ alkyl)₂, —CN, halo, or optionally substituted C₁-C₆ alkyl;
R^{3b} is H, —OH, —NH₂, —NH(C₁-C₆ alkyl), —N(C₁-C₆ alkyl)₂, —CN, halo, or optionally substituted C₁-C₆ alkyl;
R^{4a} is H, —OH, —NH₂, —NH(C₁-C₆ alkyl), —N(C₁-C₆ alkyl)₂, —CN, halo, or optionally substituted C₁-C₆ alkyl;
R^{4b} is H, —OH, —NH₂, —NH(C₁-C₆ alkyl), —N(C₁-C₆ alkyl)₂, —CN, halo, or optionally substituted C₁-C₆ alkyl;
R^{5a} is H, —OH, —NH₂, —NH(C₁-C₆ alkyl), —N(C₁-C₆ alkyl)₂, —CN, halo, or optionally substituted C₁-C₆ alkyl;
R^{5b} is H, —OH, —NH₂, —NH(C₁-C₆ alkyl), —N(C₁-C₆ alkyl)₂, —CN, halo, or optionally substituted C₁-C₆ alkyl;
R⁶ is optionally H, substituted C₁-C₂₀ alkyl, optionally substituted
C₁-C₂₀ aryl, or optionally substituted —(C₆-C₂₀ aryl)-(C₄-C₂₀ alkyl); and
M⁻ is a counter ion.

In some embodiments, R¹ is an optionally substituted C₈-C₂₀ alkyl, an optionally substituted C₈-C₂₀ alkenyl, an optionally substituted C₈-C₂₀ alkynyl, an optionally substituted —(C₆-C₂₀ aryl)-(C₄-C₂₀ alkyl), an optionally substituted —(C₄-C₂₀ alkylene)-(C₆-C₂₀ aryl), or an optionally substituted —(C₄-C₂₀ alkylene)-(C₆-C₂₀ aryl)-(C₄-C₂₀ alkyl). In other embodiments, R¹ is an optionally substituted C₈-C₂₀ alkyl. In other embodiments, R¹ is C₈-C₂₀ alkyl. In other embodiments, R¹ is an unsubstituted C₈-C₂₀ alkyl. In other embodiments, R¹ is an optionally substituted C₈-C₂₀ alkenyl. In other embodiments, R¹ is C₈-C₂₀ alkenyl. In other embodiments, R¹ is an unsubstituted C₈-C₂₀ alkenyl. In other embodiments, R¹ is an optionally substituted C₈-C₂₀ alkynyl. In other embodiments, R¹ is C₈-C₂₀ alkynyl. In other embodiments, R¹ is an unsubstituted C₈-C₂₀ alkynyl. In other embodiments, R¹ is an optionally substituted —(C₆-C₂₀ aryl)-(C₄-C₂₀ alkyl). In other embodiments, R¹ is —(C₆-C₂₀ aryl)-(C₄-C₂₀ alkyl). In other embodiments, R¹ is an unsubstituted —(C₆-C₂₀ aryl)-(C₄-C₂₀ alkyl). In other embodiments, R¹ is an optionally substituted —(C₄-C₂₀ alkylene)-(C₆-C₂₀ aryl). In other embodiments, R¹ is —(C₄-C₂₀ alkylene)-(C₆-C₂₀ aryl). In other embodiments, R¹ is an unsubstituted —(C₄-C₂₀ alkylene)-(C₆-C₂₀ aryl). In other embodiments, R¹ is an optionally substituted —(C₄-C₂₀ alkylene)-(C₆-C₂₀ aryl)-(C₄-C₂₀ alkyl). In other embodiments, R¹ is —(C₄-C₂₀ alkylene)-(C₆-C₂₀ aryl)-(C₄-C₂₀ alkyl). In other embodiments, R¹ is an unsubstituted —(C₄-C₂₀ alkylene)-(C₆-C₂₀ aryl)-(C₄-C₂₀ alkyl).

In some embodiments, R¹ is an optionally substituted C₈-C₂₀ alkyl, an optionally substituted C₈-C₂₀ alkenyl, an optionally substituted C₈-C₂₀ alkynyl, an optionally substituted —(C₆-C₁₀ aryl)-(C₄-C₂₀ alkyl), an optionally substituted —(C₄-C₂₀ alkylene)-(C₆-C₁₀ aryl), or an optionally substituted —(C₄-C₂₀ alkylene)-(C₆-C₁₀ aryl)-(C₄-C₂₀ alkyl). In other embodiments, R¹ is an optionally substituted —(C₆-C₁₀ aryl)-(C₄-C₂₀ alkyl). In other embodiments, R¹ is —(C₆-C₁₀ aryl)-(C₄-C₂₀ alkyl). In other embodiments, R¹ is an unsubstituted —(C₆-C₁₀ aryl)-(C₄-C₂₀ alkyl). In other embodiments, R¹ is an optionally substituted —(C₄-C₂₀ alkylene)-(C₆-C₁₀ aryl). In other embodiments, R¹ is —(C₄-C₂₀ alkylene)-(C₆-C₁₀ aryl). In other embodiments, R¹ is an unsubstituted —(C₄-C₂₀ alkylene)-(C₆-C₁₀ aryl). In other embodiments, R¹ is an optionally substituted —(C₄-C₂₀ alkylene)-(C₆-C₁₀ aryl)-(C₄-C₂₀ alkyl). In other embodiments, R¹ is —(C₄-C₂₀ alkylene)-(C₆-C₁₀ aryl)-(C₄-C₂₀ alkyl). In other embodiments, R¹ is an unsubstituted —(C₄-C₂₀ alkylene)-(C₆-C₁₀ aryl)-(C₄-C₂₀ alkyl).

In some embodiments, R¹ is selected from octyl, octanyl, octynyl, nonyl, nonenyl, nonynyl, decyl, decenyl, decynyl, undecyl, undecenyl, undecynyl, dodecyl, dodecenyl, dodecynyl, tridecyl, tridecenyl, tridecynyl, tetradecyl, tetradecenyl, tetradecynyl, pentadecyl, pentadecenyl, pentadecynyl, hexadecyl, hexadecenyl, hexadecynyl, heptadecyl, heptadecenyl, heptadecynyl, octadecyl, octadecenyl, octadecynyl, nonadecyl, nonadecenyl, nonadecynyl, icosyl, icosenyl, and icosynyl. In other embodiments, R¹ is octyl. In other embodiments, R¹ is octanyl. In other embodiments, R¹ is octynyl. In other embodiments, R¹ is nonyl. In other embodiments, R¹ is nonenyl. In other embodiments, R¹ is nonynyl. In other embodiments, R¹ is decyl. In other embodiments, R¹ is decenyl. In other embodiments, R¹ is decynyl. In other embodiments, R¹ is undecyl. In other embodiments, R¹ is undecenyl. In other embodiments, R¹ is undecynyl. In other embodiments, R¹ is dodecyl. In other embodiments, R¹ is dodecenyl. In other embodiments, R¹ is dodecynyl. In other embodiments, R¹ is tridecyl. In other embodiments, R¹ is tridecenyl. In other embodiments, R¹ is tridecynyl. In other embodiments, R¹ is tetradecyl. In other embodiments, R¹ is tetradecenyl. In other embodiments, R¹ is tetradecynyl. In other embodiments, R¹ is pentadecyl. In other embodiments, R¹ is pentadecenyl. In other embodiments, R¹ is pentadecynyl. In other embodiments, R¹ is hexadecyl. In other embodiments, R¹ is hexadecenyl. In other embodiments, R¹ is hexadecynyl. In other embodiments, R¹ is heptadecyl. In other embodiments, R¹ is heptadecenyl. In other embodiments, R¹ is heptadecynyl. In other embodiments, R¹ is octadecyl. In other embodiments, R¹ is octadecenyl. In other embodiments, R¹ is octadecynyl. In other embodiments, R¹ is nonadecyl. In other embodiments, R¹ is nonadecenyl. In other embodiments, R¹ is nonadecynyl. In other embodiments, R¹ is icosyl. In other embodiments, R¹ is icosenyl. In other embodiments, R¹ is icosynyl

In certain embodiments, R¹ is heptadecenyl. In certain other embodiments, R¹ is heptadec-8-enyl. In yet other embodiments, R¹ is heptadec-8-en-1-yl.

In some embodiments, R² is optionally substituted C₁-C₂₀ alkyl, optionally substituted C₂-C₂₀ hydroxyalkyl, optionally substituted —(C₁-C₂₀ alkylene)-(C₆-C₂₀ aryl), optionally substituted —(C₂-C₂₀ hydroxyalkylene)-(C₆-C₂₀ aryl), or optionally substituted —(C₂-C₂₀ hydroxyalkylene)-(C₆-C₂₀ aryl)-(C₆-C₂₀ alkyl). In other embodiments, R² is optionally substituted C₁-C₂₀ alkyl. In other embodiments, R² is C₁-C₂₀ alkyl. In other embodiments, R² is unsubstituted C₁-C₂₀ alkyl. In other embodiments, R² is optionally substituted C₂-C₂₀ hydroxyalkyl. In other embodiments, R² is optionally substituted C₂-C₂₀ β-hydroxyalkyl. In other embodiments, R² is C₂-C₂₀ hydroxyalkyl. In other embodiments, R² is C₂-C₂₀ β-hydroxyalkyl. In other embodiments, R² is unsubstituted C₂-C₂₀ hydroxyalkyl. In other embodiments, R² is unsubstituted C₂-C₂₀ β-hydroxyalkyl. In other embodiments, R² is optionally substituted —(C₁-C₂₀ alkylene)-(C₆-C₂₀ aryl). In other embodiments, R² is —(C₁-C₂₀ alkylene)-(C₆-C₂₀ aryl). In other embodiments, R² is unsubstituted —(C₁-C₂₀ alkylene)-(C₆-C₂₀ aryl). In other embodiments, R² is optionally substituted —(C₂-C₂₀ hydroxyalkylene)-(C₆-C₂₀ aryl). In other embodiments, R² is optionally substituted —(C₂-C₂₀ β-hydroxyalkylene)-(C₆-C₂₀ aryl). In other embodiments, R² is —(C₂-C₂₀ hydroxyalkylene)-(C₆-C₂₀ aryl). In other embodiments, R² is —(C₂-C₂₀ β-hydroxyalkylene)-(C₆-C₂₀ aryl). In other embodiments, R² is unsubstituted —(C₂-C₂₀ hydroxyalkylene)-(C₆-C₂₀ aryl). In other embodiments, R² is unsubstituted —(C₂-C₂₀ β-hydroxyalkylene)-(C₆-C₂₀ aryl). In other embodiments, R² is optionally substituted —(C₂-C₂₀ hydroxyalkylene)-(C₆-C₂₀ aryl)-(C₆-C₂₀ alkyl). In other embodiments, R² is optionally substituted —(C₂-C₂₀ β-hydroxyalkylene)-(C₆-C₂₀ aryl)-(C₆-C₂₀ alkyl). In other embodiments, R² is —(C₂-C₂₀ hydroxyalkylene)-(C₆-C₂₀ aryl)-(C₆-C₂₀ alkyl). In other embodiments, R² is —(C₂-C₂₀ β-hydroxyalkylene)-(C₆-C₂₀ aryl)-(C₆-C₂₀ alkyl). In other embodiments, R² is unsubstituted —(C₂-C₂₀ hydroxyalkylene)-(C₆-C₂₀ aryl)-(C₆-C₂₀ alkyl). In other embodiments, R² is unsubstituted —(C₂-C₂₀ β-hydroxyalkylene)-(C₆-C₂₀ aryl)-(C₆-C₂₀ alkyl).

In some embodiments, R² is optionally substituted C₁-C₂₀ alkyl, optionally substituted C₂-C₂₀ hydroxyalkyl, optionally substituted —(C₁-C₂₀ alkylene)-(C₆-C₁₀ aryl), optionally substituted —(C₂-C₂₀ hydroxyalkylene)-(C₆-C₁₀ aryl), or optionally substituted —(C₂-C₂₀ hydroxyalkylene)-(C₆-C₁₀ aryl)-(C₆-C₂₀ alkyl). In other embodiments, R² is optionally substituted —(C₁-C₂₀ alkylene)-(C₆-C₁₀ aryl). In other embodiments, R² is —(C₁-C₂₀ alkylene)-(C₆-C₁₀ aryl). In other embodiments, R² is unsubstituted —(C₁-C₂₀ alkylene)-(C₆-C₁₀ aryl). In other embodiments, R² is optionally substituted —(C₂-C₂₀ hydroxyalkylene)-(C₆-C₁₀ aryl). In other embodiments, R² is —(C₂-C₂₀ hydroxyalkylene)-(C₆-C₁₀ aryl). In other embodiments, R² is unsubstituted —(C₂-C₂₀ hydroxyalkylene)-(C₆-C₁₀ aryl). In other embodiments, R² is optionally substituted —(C₂-C₂₀ hydroxyalkylene)-(C₆-C₁₀ aryl)-(C₆-C₂₀ alkyl). In other embodiments, R² is —(C₂-C₂₀ hydroxyalkylene)-(C₆-C₁₀ aryl)-(C₆-C₂₀ alkyl). In other embodiments, R² is unsubstituted —(C₂-C₂₀ hydroxyalkylene)-(C₆-C₁₀ aryl)-(C₆-C₂₀ alkyl).

In some embodiments, R² is optionally substituted —CH₂CH₂(OH), optionally substituted —CH₂CH(OH)CH₃, or optionally substituted —CH₂CH(OH)-(phenyl). In other embodiments, R² is optionally substituted —CH₂CH₂(OH). In other embodiments, R² is optionally substituted —CH₂CH(OH)CH₃. In other embodiments, R² is optionally substituted —CH₂CH(OH)-(phenyl). In some embodiments, R² is —CH₂CH₂(OH), —CH₂CH(OH)CH₃, or —CH₂CH(OH)-(phenyl). In other embodiments, R² is —CH₂CH₂(OH). In other embodiments, R² is —CH₂CH(OH)CH₃. In other embodiments, R² is —CH₂CH(OH)-(phenyl). In some embodiments, R² is unsubstituted —CH₂CH₂(OH), unsubstituted —CH₂CH(OH)CH₃, or unsubstituted —CH₂CH(OH)-(phenyl). In other embodiments, R² is unsubstituted —CH₂CH₂(OH). In other embodiments, R² is unsubstituted —CH₂CH(OH)CH₃. In other embodiments, R² is unsubstituted —CH₂CH(OH)-(phenyl).

In some embodiments, R^{3a} is H, —OH, —NH₂, —NH(C₁-C₆ alkyl), —N(C₁-C₆ alkyl)₂, —CN, halo, or optionally substituted C₁-C₆ alkyl. In other embodiments, R^{3a} is H. In other embodiments, R^{3a} is —OH. In other embodiments, R^{3a} is —NH₂. In other embodiments, R^{3a} is —NH(C₁-C₆ alkyl). In other embodiments, R^{3a} is —N(C₁-C₆ alkyl)₂. In other embodiments, R^{3a} is —CN. In other embodiments, R^{3a} is halo. In other embodiments, R^{3a} is optionally substituted C₁-C₆ alkyl. In other embodiments, R^{3a} is C₁-C₆ alkyl. In other embodiments, R^{3a} is unsubstituted C₁-C₆ alkyl.

In some embodiments, R^{3b} is H, —OH, —NH₂, —NH(C₁-C₆ alkyl), —N(C₁-C₆ alkyl)₂, —CN, halo, or optionally substituted C₁-C₆ alkyl. In other embodiments, R^{3b} is H. In other embodiments, R^{3b} is —OH. In other embodiments, R^{3b} is —NH₂. In other embodiments, R^{3b} is —NH(C₁-C₆ alkyl). In other embodiments, R^{3b} is —N(C₁-C₆ alkyl)₂. In other embodiments, R^{3b} is —CN. In other embodiments, R^{3b} is halo. In other embodiments, R^{3b} is optionally substituted C₁-C₆ alkyl. In other embodiments, R^{3b} is C₁-C₆ alkyl. In other embodiments, R^{3b} is unsubstituted C₁-C₆ alkyl.

In some embodiments, R^{4a} is H, —OH, —NH₂, —NH(C₁-C₆ alkyl), —N(C₁-C₆ alkyl)₂, —CN, halo, or optionally substituted C₁-C₆ alkyl. In other embodiments, R^{4a} is H. In other embodiments, R^{4a} is —OH. In other embodiments, R^{4a} is —NH₂. In other embodiments, R^{4a} is —NH(C₁-C₆ alkyl). In other embodiments, R^{4a} is —N(C₁-C₆ alkyl)₂. In other embodiments, R^{4a} is —CN. In other embodiments, R^{4a} is halo. In other embodiments, R^{4a} is optionally substituted C₁-C₆ alkyl. In other embodiments, R^{4a} is C₁-C₆ alkyl. In other embodiments, R^{4a} is unsubstituted C₁-C₆ alkyl.

In some embodiments, R^{4b} is H, —OH, —NH₂, —NH(C₁-C₆ alkyl), —N(C₁-C₆ alkyl)₂, —CN, halo, or optionally substituted C₁-C₆ alkyl. In other embodiments, R^{4b} is H. In other embodiments, R^{4b} is —OH. In other embodiments, R^{4b} is —NH₂. In other embodiments, R^{4b} is —NH(C₁-C₆ alkyl). In other embodiments, R^{4b} is —N(C₁-C₆ alkyl)₂. In other embodiments, R^{4b} is —CN. In other embodiments, R^{4b} is halo. In other embodiments, R^{4b} is optionally substituted C₁-C₆ alkyl. In other embodiments, R^{4b} is C₁-C₆ alkyl. In other embodiments, R^{4b} is unsubstituted C₁-C₆ alkyl.

In some embodiments, M⁻ is selected from sulfate, phosphate, nitrate, sulfonate, iodide, fluoride, chloride, gluconate, allarate, altarate, altrarate, altronate, arabinarate, arabinonate, citrate, dihomocitrate, fructuronate, fuconate, fumarate, galactarate, galactonate, galacturonate, glucarate, glucoheptonate, gluconate, glucuronate, gulonate, homocitrate, homoisocitrate, idarate, idonate, iduronate, isocitrate, mannarate, mannonate, malate, tartarate, octulosonate, rhamnonate, ribonate, tagaturonate, xylonate, xyluronate, tartarate, tatronate, glycerate, malonate, pantoate, or a combination thereof. In other embodiments, M⁻ is sulfate. In other embodiments, M⁻ is phosphate. In other embodiments, M⁻ is nitrate. In other embodiments, M⁻ is sulfonate. In other embodiments, M⁻ is iodide. In other embodiments, M⁻ is fluoride. In other embodiments, M⁻ is chloride. In other embodiments, M⁻ is gluconate. In other embodiments, M⁻ is allarate. In other embodiments, M⁻ is altarate. In other embodiments, M⁻ is altrarate. In other embodiments, M⁻ is altronate. In other embodiments, M⁻ is arabinarate. In other embodiments, M⁻ is arabinonate. In other embodiments, M⁻ is citrate. In other embodiments, M⁻ is dihomocitrate. In other embodiments, M⁻ is fructuronate. In other embodiments, M⁻ is fuconate. In other embodiments, M⁻ is fumarate. In other embodiments, M⁻ is galactarate. In other embodiments, M⁻ is galactonate. In other embodiments, M⁻ is galacturonate. In other embodiments, M⁻ is glucarate. In other embodiments, M⁻ is glucoheptonate. In other embodiments, M⁻ is gluconate. In other embodiments, M⁻ is glucoronate. In other embodiments, M⁻ is gulonate. In other embodiments, M⁻ is homocitrate. In other embodiments, M⁻ is homoisocitrate. In other embodiments, M⁻ is idarate. In other embodiments, M⁻ is idonate. In other embodiments, M⁻ is iduronate. In other embodiments, M⁻ is isocitrate. In other embodiments, M⁻ is mannarate. In other embodiments, M⁻ is mannonate. In other embodiments, M⁻ is malate. In other embodiments, M⁻ is tartarate. In other embodiments, M⁻ is octulosonate. In other embodiments, M⁻ is rhamnonate. In other embodiments, M⁻ is ribonate. In other embodiments, M⁻ is tagaturonate. In other embodiments, M⁻ is xylonate. In other embodiments, M⁻ is xyluronate. In other embodiments, M⁻ is tartarate. In other embodiments, M⁻ is tatronate. In other embodiments, M⁻ is glycerate. In other embodiments, M⁻ is malonate. In other embodiments, M⁻ is pantoate.

In some embodiments, the compound of formula (I) is selected from:

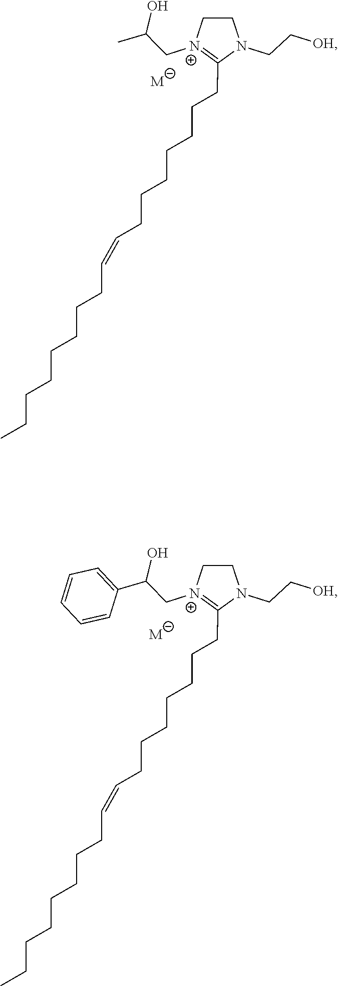

and combinations thereof, wherein M⁻ is as defined above.

In some embodiments, the compound of formula (I) is:

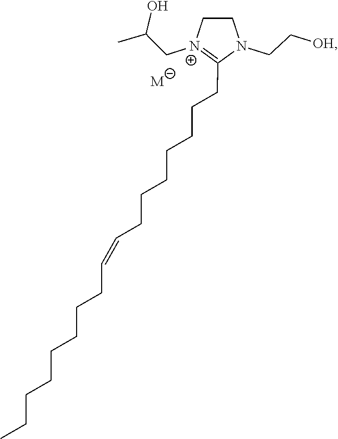

wherein M⁻ is as defined above.

In some embodiments, the compound of formula (I) is:

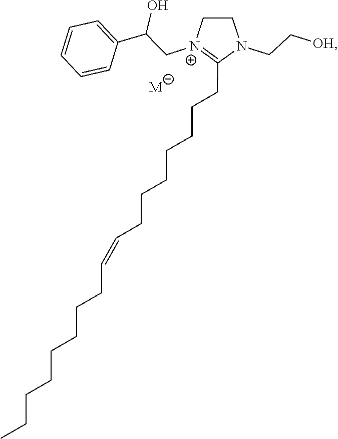

wherein M⁻ is as defined above.

In some embodiments, the compound of formula (I) is selected from:

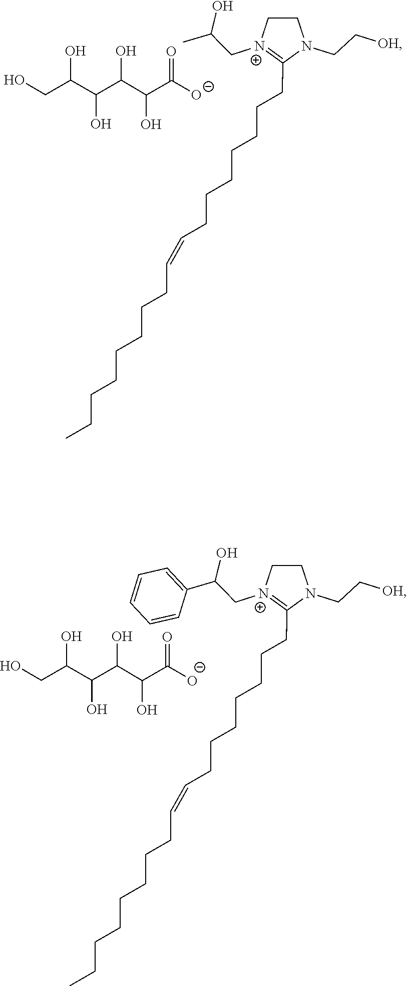 [SMILES: O=C([O-])C(O)C(O)C(O)C(O)CO ; CCCCCCCC/C=C\CCCCCCCC1=[N+](CC(C)O)CCN1CCO ; O=C([O-])C(O)C(O)C(O)C(O)CO ; CCCCCCCC/C=C\CCCCCCCC1=[N+](CC(O)c2ccccc2)CCN1CCO]

and combinations thereof.

In some embodiments, the compound of formula (I) is:

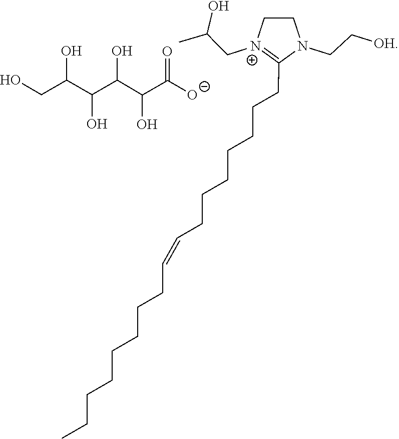 [SMILES: O=C([O-])C(O)C(O)C(O)C(O)CO ; CCCCCCCC/C=C\CCCCCCCC1=[N+](CC(C)O)CCN1CCO]

In some embodiments, the compound of formula (I) is:

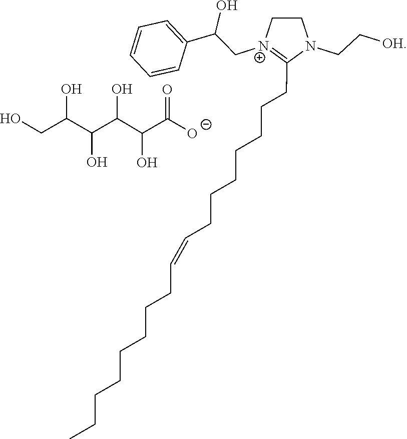 [SMILES: O=C([O-])C(O)C(O)C(O)C(O)CO ; CCCCCCCC/C=C\CCCCCCCC1=[N+](CC(O)c2ccccc2)CCN1CCO]

A method of reducing corrosion of a metal surface in contact with an aqueous medium is also provided. In some aspects, the method includes adding a compound of formula (I), as described herein, to the aqueous medium and/or the metal surface. In other aspects, the method includes adding a corrosion inhibitor composition that includes a compound of formula (I), as described herein, to the aqueous medium and/or metal surface.

The aqueous medium may include one or more corrosive agents, such as a brine, an acid, (e.g., an organic acid), carbon dioxide, and hydrogen sulfide. The compositions and methods of the present disclosure are useful for inhibiting corrosion of metal surfaces in contact with any type of corrodent in the medium, such as metal cations, metal complexes, metal chelates, organometallic complexes, aluminum ions, ammonium ions, barium ions, chromium ions, cobalt ions, cuprous ions, cupric ions, calcium ions, ferrous ions, ferric ions, hydrogen ions, magnesium ions, manganese ions, molybdenum ions, nickel ions, potassium ions, sodium ions, strontium ions, titanium ions, uranium ions, vanadium ions, zinc ions, bromide ions, carbonate ions, chlorate ions, chloride ions, chlorite ions, dithionate ions, fluoride ions, hypochlorite ions, iodide ions, nitrate ions, nitrite ions, oxide ions, perchlorate ions, peroxide ions, phosphate ions, phosphite ions, sulfate ions, sulfide ions, sulfite ions, hydrogen carbonate ions, hydrogen phosphate ions, hydrogen phosphite ions, hydrogen sulfate ions, hydrogen sulfite ions, an acid, such as carbonic acid, hydrochloric acid, nitric acid, sulfuric acid, nitrous acid, sulfurous acid, a peroxy acid, or phosphoric acid, ammonia, bromine, carbon dioxide, chlorine, chlorine dioxide, fluorine, hydrogen chloride, hydrogen sulfide, iodine, nitrogen dioxide, nitrogen monoxide, oxygen, ozone, sulfur dioxide, hydrogen peroxide, polysaccharides, metal oxides, sands, clays, silicon dioxide, titanium dioxide, muds, insoluble inorganic and/or organic particulates, an oxidizing agent, a chelating agent, an alcohol, and any combination of the foregoing.

The corrosion inhibitor compositions and methods disclosed herein provide corrosion protection for any metal, such as metallic-chrome steel, ferritic-alloy steel, austenitic-steel, precipitation-hardened steel, high-nickel steel, carbon steel, or a combination thereof.

The presently disclosed compositions and methods are useful for inhibiting corrosion of surfaces comprising any metal or combination of metals. In some aspects, the metal surface comprises steel, such as stainless steel or carbon steel. In some aspects, the metal surface comprises iron, aluminum, zinc, chromium, manganese, nickel, tungsten, molybdenum, titanium, vanadium, cobalt, niobium, or copper. The metal surface may also comprise any combination of the foregoing metals and/or any one or more of boron, phosphorus, sulfur, silicon, oxygen, and nitrogen. In some aspects, a pipe, heat exchanger, storage vessel, or a tank (e.g., railroad tank car or a tank truck/tanker) comprises the metallic surface.

While a compound of formula (I) or a salt thereof, as disclosed herein, can be added to an aqueous medium at any dosage rate, an example of a dosage rate includes about 0.01 ppm to about 50,000 ppm. In some embodiments, the compound of formula (I), or a salt thereof, is added to an aqueous medium at a dosage rate of about 5 ppm to about 50,000 ppm. In other aspects, the compound of formula (I), or a salt thereof, is added to an aqueous medium at a dosage rate of about 5 ppm to about 20,000 ppm. In other aspects, the compound of formula (I), or a salt thereof, is added to an aqueous medium at a dosage rate of about 5 ppm to about 10,000 ppm. In other aspects, the compound of formula (I), or a salt thereof, is added to an aqueous medium at a dosage rate of about 5 ppm to about 5,000 ppm. In other aspects, the compound of formula (I), or a salt thereof, is added to an aqueous medium at a dosage rate of about 5 ppm to about 2,000 ppm. In other aspects, the compound of formula (I), or a salt thereof, is added to an aqueous medium at a dosage rate of about 5 ppm to about 1,000 ppm. In other aspects, the compound of formula (I), or a salt thereof, is added to an aqueous medium at a dosage rate of about 5 ppm to about 500 ppm. In other aspects, the compound of formula (I), or a salt thereof, is added to an aqueous medium at a dosage rate of about 5 ppm to about 50 ppm. In other aspects, the compound of formula (I), or a salt thereof, is added to an aqueous medium at a dosage rate of about 5 ppm to about 25 ppm. In other aspects, the compound of formula (I), or a salt thereof, is added to an aqueous medium at a dosage rate of about 5 ppm to about 10 ppm.

In certain aspects, the compound of formula (I), or a salt thereof, is added to an aqueous medium at a dosage rate of about 5 ppm. In certain aspects, the compound of formula (I), or a salt thereof, is added to an aqueous medium at a dosage rate of about 10 ppm. In certain aspects, the compound of formula (I), or a salt thereof, is added to an aqueous medium at a dosage rate of about 25 ppm. In certain aspects, the compound of formula (I), or a salt thereof, is added to an aqueous medium at a dosage rate of about 50 ppm. In certain aspects, the compound of formula (I), or a salt thereof, is added to an aqueous medium at a dosage rate of about 100 ppm. In certain aspects, the compound of formula (I), or a salt thereof, is added to an aqueous medium at a dosage rate of about 250 ppm. In certain aspects, the compound of formula (I), or a salt thereof, is added to an aqueous medium at a dosage rate of about 500 ppm. In certain aspects, the compound of formula (I), or a salt thereof, is added to an aqueous medium at a dosage rate of about 750 ppm. In certain aspects, the compound of formula (I), or a salt thereof, is added to an aqueous medium at a dosage rate of about 1,000 ppm. In certain aspects, the compound of formula (I), or a salt thereof, is added to an aqueous medium at a dosage rate of about 5,000 ppm. In certain aspects, the compound of formula (I), or a salt thereof, is added to an aqueous medium at a dosage rate of about 10,000 ppm.

While a corrosion inhibitor composition and/or formulation as disclosed herein can be added to an aqueous medium at any dosage rate, an example of a dosage rate is about 0.01 ppm to about 50,000 ppm. In some embodiments, the corrosion inhibitor composition and/or formulation is added to an aqueous medium at a dosage rate of about 5 ppm to about 50,000 ppm. In other aspects, the corrosion inhibitor composition and/or formulation is added to an aqueous medium at a dosage rate of about 5 ppm to about 20,000 ppm. In other aspects, the corrosion inhibitor composition and/or formulation is added to an aqueous medium at a dosage rate of about 5 ppm to about 10,000 ppm. In other aspects, the corrosion inhibitor composition and/or formulation is added to an aqueous medium at a dosage rate of about 5 ppm to about 5,000 ppm. In other aspects, the corrosion inhibitor composition and/or formulation is added to an aqueous medium at a dosage rate of about 5 ppm to about 2,000 ppm. In other aspects, the corrosion inhibitor composition and/or formulation is added to an aqueous medium at a dosage rate of about 5 ppm to about 1,000 ppm. In other aspects, the corrosion inhibitor composition and/or formulation is added to an aqueous medium at a dosage rate of about 5 ppm to about 500 ppm. In other aspects, the corrosion inhibitor composition and/or formulation is added to an aqueous medium at a dosage rate of about 5 ppm to about 50 ppm. In other aspects, the corrosion inhibitor composition and/or formulation is added to an aqueous medium at a dosage rate of about 5 ppm to about 25 ppm. In other aspects, the corrosion inhibitor composition and/or formulation is added to an aqueous medium at a dosage rate of about 5 ppm to about 10 ppm.

In certain aspects, the corrosion inhibitor composition and/or formulation is added to an aqueous medium at a dosage rate of about 5 ppm. In certain aspects, the corrosion inhibitor composition and/or formulation is added to an aqueous medium at a dosage rate of about 10 ppm. In certain aspects, the corrosion inhibitor composition and/or formulation is added to an aqueous medium at a dosage rate of about 25 ppm. In certain aspects, the corrosion inhibitor composition and/or formulation is added to an aqueous medium at a dosage rate of about 50 ppm. In certain aspects, the corrosion inhibitor composition and/or formulation is added to an aqueous medium at a dosage rate of about 100 ppm. In certain aspects, the corrosion inhibitor composition and/or formulation is added to an aqueous medium at a dosage rate of about 250 ppm. In certain aspects, the corrosion inhibitor composition and/or formulation is added to an aqueous medium at a dosage rate of about 500 ppm. In certain aspects, the corrosion inhibitor composition and/or formulation is added to an aqueous medium at a dosage rate of about 750 ppm. In certain aspects, the corrosion inhibitor composition and/or formulation is added to an aqueous medium at a dosage rate of about 1,000 ppm. In certain aspects, the corrosion inhibitor composition and/or formulation is added to an aqueous medium at a dosage rate of about 3,000 ppm. In certain aspects, the corrosion inhibitor composition and/or formulation is added to an aqueous medium at a dosage rate of about 5,000 ppm.

The metal corrosion rate provided by a corrosion inhibitor composition and/or formulation as disclosed herein is not limited. In certain embodiments, a corrosion inhibitor composition and/or formulation as disclosed herein provides a metal corrosion rate that is acceptable according to industry standards, e.g., about 0.2 mpy or less. In certain aspects, a corrosion inhibitor composition and/or formulation as disclosed herein provides a metal corrosion rate of about 0.1 mpy or less. In additional aspects, a corrosion inhibitor composition and/or formulation as disclosed herein provides a metal corrosion rate of about 0.1 mpy or less, about 0.05 mpy or less, about 0.04 mpy or less, about 0.03 mpy or less, about 0.02 mpy or less, about 0.01 mpy or less, about 0.005 mpy or less, or about 0.002 mpy or less.

The corrosion inhibitor compositions and/or formulations as disclosed herein can be used to inhibit corrosion of metal in an aqueous system having any pH. In certain aspects, a corrosion inhibitor composition and/or formulation as disclosed herein is added to an aqueous system having a pH of from about 3 to about 12, from about 3 to about 11, from about 3 to about 10, from about 3 to about 9, from about 3 to about 8, from about 3 to about 7, from about 3 to about 6, from about 3 to about 5, from about 3 to about 4, from about 4 to about 12, from about 5 to about 12, from about 6 to about 12, from about 7 to about 12, from about 8 to about 12, from about 9 to about 12, from about 10 to about 12, from about 11 to about 12, from about 6 to about 9, from about 6 to about 8, or from about 6 to about 7.

In certain aspects, a corrosion inhibitor composition and/or formulation as disclosed herein inhibits metal corrosion when added to an aqueous medium even if the medium comprises a biocide, such as a non-halogen-containing oxidizing biocide including, but not limited to, peroxides (e.g., hydrogen peroxide), persulfates, permanganates, and peracetic acids.

The corrosion inhibitor compositions and/or formulations as disclosed herein are contacted with a metal surface by any suitable method. In certain embodiments, a corrosion inhibitor composition (or solution comprising the composition) and/or formulation as disclosed herein is contacted with a metal surface by immersion, spraying, or other coating techniques. In certain embodiments, a corrosion inhibitor composition and/or formulation is introduced into the aqueous medium by any conventional method, such as manually or automatically using a chemical injection pump, and is fed into the aqueous medium on either a periodic or continuous basis.

In certain aspects, if a corrosion inhibitor composition and/or formulation as disclosed herein is relatively insoluble in water, the composition may be made soluble by forming an organic or inorganic salt of one or more of the compounds within the composition/formulation. Thus, in certain aspects, a corrosion inhibitor composition and/or formulation as disclosed herein comprises a water-soluble salt of one or more of the compounds disclosed herein. In certain aspects, a corrosion inhibitor composition and/or formulation as disclosed herein is added as a solution in a water-miscible co-solvent including, but not limited to, acetone, methanol, ethanol, propanol, formic acid, formamide, propylene glycol, or ethylene glycol. In certain embodiments, a co-solvent is used to achieve maximum solubility of a corrosion inhibitor composition and/or formulation as disclosed herein in the aqueous system. In certain aspects, low molecular weight polyethylene glycol, polypropylene glycol, a surfactant (e.g., organic sulfonic acid), or combinations thereof are used to increase the solubility of a corrosion inhibitor composition and/or formulation as disclosed herein.

Those skilled in the art will appreciate that the corrosion inhibitor compositions and/or formulations disclosed herein can be added to an aqueous medium alone or in combination with other corrosion inhibitors or treatment chemicals. Multiple corrosion inhibitors can be dosed as a combined corrosion inhibitor formulation or each corrosion inhibitor can be added separately, including two or more corrosion inhibitor compositions as disclosed herein. Moreover, the corrosion inhibitor compositions and/or formulations disclosed herein can be added to an aqueous system in combination with a variety of additional corrosion inhibitors including, but not limited to, azoles, orthophosphate, polyphosphates, phosphonates, molybdates, silicates, oximes, imidazolines, and nitrites.

The corrosion inhibitor compositions and/or formulations disclosed herein also can be added to an aqueous system in combination with a variety of additional additives, such as treatment polymers, anti-microbial agents, anti-scaling agents (i.e., antiscalants), colorants, fillers, buffers, surfactants, viscosity modifiers, chelating agents, dispersants, deodorants, masking agents, oxygen scavengers, and indicator dyes.

In some embodiments, the corrosion inhibitor composition of the disclosure further includes an antiscalant.

In some embodiments, the antiscalant is selected from allaric acid, altaric acid, altraric acid, altronic acid, arabinaric acid, arabinonic acid, citric acid, dihomocitric acid, fructuronic acid, fuconic acid, fumaric acid, galactaric acid, galactonic acid, galacturonic acid, glucaric acid, glucoheptonic acid, gluconic acid, glucuronic acid, gulonic acid, homocitric acid, homoisocitric acid, idaric acid, idonic acid, iduronic acid, isocitric acid, mannaric acid, mannonic acid, malic acid, tartaric acid, octulosonic acid, rhamnonic acid, ribonic acid, tagaturonic acid, xylonic acid, xyluronic acid, tartaric acid, tatronic acid, glyceric acid, malonic acid, and pantoic acid, or a salt thereof. In other embodiments, the antiscalant is allaric acid or a salt thereof. In other embodiments, the antiscalant is altaric acid or a salt thereof. In other embodiments, the antiscalant is altraric acid or a salt thereof. In other embodiments, the antiscalant is altronic acid or a salt thereof. In other embodiments, the antiscalant is arabinaric acid or a salt thereof. In other embodiments, the antiscalant is arabinonic acid or a salt thereof. In other embodiments, the antiscalant is citric acid or a salt thereof. In other embodiments, the antiscalant is dihomocitric acid or a salt thereof. In other embodiments, the antiscalant is fructuronic acid or a salt thereof. In other embodiments, the antiscalant is fuconic acid or a salt thereof. In other embodiments, the antiscalant is fumaric acid or a salt thereof. In other embodiments, the antiscalant is galactaric acid or a salt thereof. In other embodiments, the antiscalant is galactonic acid or a salt thereof. In other embodiments, the antiscalant is galacturonic acid or a salt thereof. In other embodiments, the antiscalant is glucaric acid or a salt thereof. In other embodiments, the antiscalant is glucoheptonic acid or a salt thereof. In other embodiments, the antiscalant is gluconic acid or a salt thereof. In other embodiments, the antiscalant is glucuronic acid or a salt thereof. In other embodiments, the antiscalant is gulonic acid or a salt thereof. In other embodiments, the antiscalant is homocitric acid or a salt thereof. In other embodiments, the antiscalant is homoisocitric acid or a salt thereof. In other embodiments, the antiscalant is idaric acid or a salt thereof. In other embodiments, the antiscalant is idonic acid or a salt thereof. In other embodiments, the antiscalant is iduronic acid or a salt thereof. In other embodiments, the antiscalant is isocitric acid or a salt thereof. In other embodiments, the antiscalant is mannaric acid or a salt thereof. In other embodiments, the antiscalant is mannonic acid or a salt thereof. In other embodiments, the antiscalant is malic acid or a salt thereof. In other embodiments, the antiscalant is tartaric acid or a salt thereof. In other embodiments, the antiscalant is octulosonic acid or a salt thereof. In other embodiments, the antiscalant is rhamnonic acid or a salt thereof. In other embodiments, the antiscalant is ribonic acid or a salt thereof. In other embodiments, the antiscalant is tagaturonic acid or a salt thereof. In other embodiments, the antiscalant is xylonic acid or a salt thereof. In other embodiments, the antiscalant is xyluronic acid or a salt thereof. In other embodiments, the antiscalant is tartaric acid or a salt thereof. In other embodiments, the antiscalant is tatronic acid or a salt thereof. In other embodiments, the antiscalant is glyceric acid or a salt thereof. In other embodiments, the antiscalant is malonic acid or a salt thereof. In other embodiments, the antiscalant is pantoic acid or a salt thereof.

The corrosion inhibitor composition may comprise various amounts of each component. For example, in some embodiments, the composition may comprise from about 99% by weight water and about 1% by weight of one or more corrosion inhibitor compounds of formula (I) to about 50% by weight water and 50% by weight of one or more corrosion inhibitor compounds of formula (I). In other embodiments, the composition may comprise from about 99% by weight water and about 1% by weight of one or more corrosion inhibitor compounds of formula (I). In other embodiments, the composition may comprise from about 95% by weight water and about 5% by weight of one or more corrosion inhibitor compounds of formula (I). In other embodiments, the composition may comprise from about 90% by weight water and about 10% by weight of one or more corrosion inhibitor compounds of formula (I). In other embodiments, the composition may comprise from about 85% by weight water and about 15% by weight of one or more corrosion inhibitor compounds of formula (I). In other embodiments, the composition may comprise from about 80% by weight water and about 20% by weight of one or more corrosion inhibitor compounds of formula (I). In other embodiments, the composition may comprise from about 75% by weight water and about 25% by weight of one or more corrosion inhibitor compounds of formula (I). In other embodiments, the composition may comprise from about 70% by weight water and about 30% by weight of one or more corrosion inhibitor compounds of formula (I). In other embodiments, the composition may comprise from about 65% by weight water and about 35% by weight of one or more corrosion inhibitor compounds of formula (I). In other embodiments, the composition may comprise from about 60% by weight water and about 40% by weight of one or more corrosion inhibitor compounds of formula (I). In other embodiments, the composition may comprise from about 55% by weight water and about 45% by weight of one or more corrosion inhibitor compounds of formula (I). In other embodiments, the composition may comprise from about 50% by weight water and about 50% by weight of one or more corrosion inhibitor compounds of formula (I).

The corrosion inhibitor compositions and/or formulations as disclosed herein can be added to an aqueous medium in any form. In certain aspects, a corrosion inhibitor composition and/or formulation is added to an aqueous medium as a dried solid. In certain embodiments, a corrosion inhibitor composition and/or formulation is added to an aqueous medium as a solution in a co-solvent miscible with water. In certain embodiments, a corrosion inhibitor composition and/or formulation as disclosed herein is added to an aqueous medium as an aqueous solution.

In certain aspects, a corrosion inhibitor composition and/or formulation as disclosed herein is added to a medium in an oil or natural gas recovery system, an oil or natural gas transportation system, an oil or natural gas storage system, an oil or natural gas processing system, an aqueous system that recirculates water, and/or an aqueous system that has stagnant water.

A process for preparing a compound of formula (I-A) is additionally provided. The process for preparing a compound of formula (I-A) includes contacting a compound of formula (I-B):

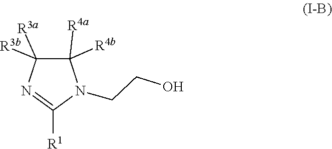

with a compound of formula (I-C):

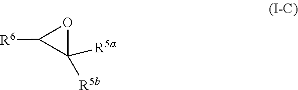

wherein:
R¹ is optionally substituted C₈-C₂₀ alkyl, an optionally substituted
C₈-C₂₀ alkenyl with one to multiple vinylic moieties, or an optionally substituted C₈-C₂₀ alkynyl with one to multiple vinylic moieties, an optionally substituted —(C₆-C₂₀ aryl)-(C₄-C₂₀ alkyl), an optionally substituted
—(C₄-C₂₀ alkylene)-(C₆-C₂₀ aryl), or an optionally substituted
—(C₄-C₂₀ alkylene)-(C₆-C₂₀ aryl)-(C₄-C₂₀ alkyl);
R^{3a} is H, —OH, —NH₂, —NH(C₁-C₆ alkyl), —N(C₁-C₆ alkyl)₂, —CN, halo, or optionally substituted C₁-C₆ alkyl;
R^{3b} is H, —OH, —NH₂, —NH(C₁-C₆ alkyl), —N(C₁-C₆ alkyl)₂, —CN, halo, or optionally substituted C₁-C₆ alkyl;
R^{4a} is H, —OH, —NH₂, —NH(C₁-C₆ alkyl), —N(C₁-C₆ alkyl)₂, —CN, halo, or optionally substituted C₁-C₆ alkyl;
R^{4b} is H, —OH, —NH₂, —NH(C₁-C₆ alkyl), —N(C₁-C₆ alkyl)₂, —CN, halo, or optionally substituted C₁-C₆ alkyl; and
R^{5a} is H, —OH, —NH₂, —NH(C₁-C₆ alkyl), —N(C₁-C₆ alkyl)₂, —CN, halo, or optionally substituted C₁-C₆ alkyl;
R^{5b} is H, —OH, —NH₂, —NH(C₁-C₆ alkyl), —N(C₁-C₆ alkyl)₂, —CN, halo, or optionally substituted C₁-C₆ alkyl;
R⁶ is optionally substituted C₁-C₆ alkyl or optionally substituted C₆-C₁₀ aryl; and
M⁻ is a counter ion.

In some aspects, the compound of formula (I-B) is contacted with the compound of formula (I-C) in the presence of an acid. One of ordinary skill in the art will appreciate that there are many such acids that are compatible with the process and would be capable of selecting such an acid without the burden of undue experimentation. In some aspects, the acid is selected from gluconic acid, allaric acid, altaric acid, altraric acid, altronic acid, arabinaric acid, arabinonic acid, citric acid, dihomocitric acid, fructuronic acid, fuconic acid, fumaric acid, galactaric acid, galactonic acid, galacturonic acid, glucaric acid, glucoheptonic acid, gluconic acid, glucuronic acid, gulonic acid, homocitric acid, homoisocitric acid, idaric acid, idonic acid, iduronic acid, isocitric acid, mannaric acid, mannonic acid, malic acid, tartaric acid, octulosonic acid, rhamnonic acid, ribonic acid, tagaturonic acid, xylonic acid, xyluronic acid, tartaric acid, tatronic acid, glyceric acid, malonic acid, pantoic acid, or a combination thereof. In other aspects, the acid is gluconic acid. In other aspects, the acid is allaric acid. In other aspects, the acid is altaric acid. In other aspects, the acid is altraric acid. In other aspects, the acid is altronic acid. In other aspects, the acid is arabinaric acid. In other aspects, the acid is arabinonic acid. In other aspects, the acid is citric acid. In other aspects, the acid is dihomocitric acid. In other aspects, the acid is fructuronic acid. In other aspects, the acid is fuconic acid. In other aspects, the acid is fumaric acid. In other aspects, the acid is galactaric acid. In other aspects, the acid is galactonic acid. In other aspects, the acid is galacturonic acid. In other aspects, the acid is glucaric acid. In other aspects, the acid is glucoheptonic acid. In other aspects, the acid is gluconic acid. In other aspects, the acid is glucuronic acid. In other aspects, the acid is gulonic acid. In other aspects, the acid is homocitric acid. In other aspects, the acid is homoisocitric acid. In other aspects, the acid is idaric acid. In other aspects, the acid is idonic acid. In other aspects, the acid is iduronic acid. In other aspects, the acid is isocitric acid. In other aspects, the acid is mannaric acid. In other aspects, the acid is mannonic acid. In other aspects, the acid is malic acid. In other aspects, the acid is tartaric acid. In other aspects, the acid is octulosonic acid. In other aspects, the acid is rhamnonic acid. In other aspects, the acid is ribonic acid. In other aspects, the acid is tagaturonic acid. In other aspects, the acid is xylonic acid. In other aspects, the acid is xyluronic acid. In other aspects, the acid is tartaric acid. In other aspects, the acid is tatronic acid. In other aspects, the acid is glyceric acid. In other aspects, the acid is malonic acid. In other aspects, the acid is pantoic acid.

In some aspects, the compound of formula (I-B) is contacted with the compound of formula (I-C) in the presence of heat. One of ordinary skill in the art will appreciate that there are many reaction temperatures compatible with the process and would be capable of selecting such a reaction temperature without the burden of undue experimentation. In some aspects, the compound of formula (I-B) is contacted with the compound of formula (I-C) at a temperature in the range of about 70° C. to about 150° C. In other aspects, the compound of formula (I-B) is contacted with the compound of formula (I-C) at a temperature the range of about 75° C. to about 145° C. In other aspects, the compound of formula (I-B) is contacted with the compound of formula (I-C) at a temperature the range of about 80° C. to about 140° C. In other aspects, the compound of formula (I-B) is contacted with the compound of formula (I-C) at a temperature the range of about 85° C. to about 140° C. In other aspects, the compound of formula (I-B) is contacted with the compound of formula (I-C) at a temperature the range of about 90° C. to about 135° C. In other aspects, the compound of formula (I-B) is contacted with the compound of formula (I-C) at a temperature the range of about 90° C. to about 130° C. In other aspects, the compound of formula (I-B) is contacted with the compound of formula (I-C) at a temperature the range of about 90° C. to about 125° C. In other aspects, the compound of formula (I-B) is contacted with the compound of formula (I-C) at a temperature the range of about 90° C. to about 120° C. In other aspects, the compound of formula (I-B) is contacted with the compound of formula (I-C) at a temperature the range of about 90° C. to about 115° C. In other aspects, the compound of formula (I-B) is contacted with the compound of formula (I-C) at a temperature the range of about 90° C. to about 110° C. In other aspects, the compound of formula (I-B) is contacted with the compound of formula (I-C) at a temperature the range of about 90° C. to about 105° C. In other aspects, the compound of formula (I-B) is contacted with the compound of formula (I-C) at a temperature the range of about 90° C. to about 100° C. In other aspects, the compound of formula (I-B) is contacted with the compound of formula (I-C) at a temperature of about 95° C. In other aspects, the compound of formula (I-B) is contacted with the compound of formula (I-C) under reflux at a temperature of about 95° C.

In some aspects, the process for preparing a compound of formula (I) further includes contacting a compound of formula (I-D):

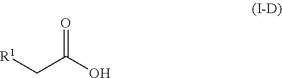

with a compound of formula (I-E):

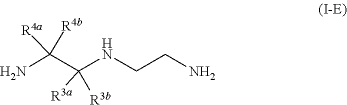

to produce the compound of formula (I-B).

In some aspects, M⁻ is selected from sulfate, phosphate, nitrate, sulfonate, iodide, fluoride, chloride, gluconate, allarate, altarate, altrarate, altronate, arabinarate, arabinonate, citrate, dihomocitrate, fructuronate, fuconate, fumarate, galactarate, galactonate, galacturonate, glucarate, glucoheptonate, gluconate, glucuronate, gulonate, homocitrate, homoisocitrate, idarate, idonate, iduronate, isocitrate, mannarate, mannonate, malate, tartarate, octulosonate, rhamnonate, ribonate, tagaturonate, xylonate, xyluronate, tartarate, tatronate, glycerate, malonate, pantoate, or a combination thereof. In other aspects, M⁻ is sulfate. In other aspects, M⁻ is phosphate. In other aspects, M⁻ is nitrate. In other aspects, M⁻ is sulfonate. In other aspects, M⁻ is iodide. In other aspects, M⁻ is fluoride. In other aspects, M⁻ is chloride. In other aspects, M⁻ is gluconate. In other aspects, M⁻ is allarate. In other aspects, M⁻ is altarate. In other aspects, M⁻ is altrarate. In other aspects, M⁻ is altronate. In other aspects, M⁻ is arabinarate. In other aspects, M⁻ is arabinonate. In other aspects, M⁻ is citrate. In other aspects, M⁻ is dihomocitrate. In other aspects, M⁻ is fructuronate. In other aspects, M⁻ is fuconate. In other aspects, M⁻ is fumarate. In other aspects, M⁻ is galactarate. In other aspects, M⁻ is galactonate. In other aspects, M⁻ is galacturonate. In other aspects, M⁻ is glucarate. In other aspects, M⁻ is glucoheptonate. In other aspects, M⁻ is gluconate. In other aspects, M⁻ is glucoronate. In other aspects, M⁻ is gulonate. In other aspects, M⁻ is homocitrate. In other aspects, M⁻ is homoisocitrate. In other aspects, M⁻ is idarate. In other aspects, M⁻ is idonate. In other aspects, M⁻ is iduronate. In other aspects, M⁻ is isocitrate. In other aspects, M⁻ is mannarate. In other aspects, M⁻ is mannonate. In other aspects, M⁻ is malate. In other aspects, M⁻ is tartarate. In other aspects, M⁻ is octulosonate. In other aspects, M⁻ is rhamnonate. In other aspects, M⁻ is ribonate. In other aspects, M⁻ is tagaturonate. In other aspects, M⁻ is xylonate. In other aspects, M⁻ is xyluronate. In other aspects, M⁻ is tartarate. In other aspects, M⁻ is tatronate. In other aspects, M⁻ is glycerate. In other aspects, M⁻ is malonate. In other aspects, M⁻ is pantoate.

### EXAMPLES

The foregoing may be better understood by reference to the following examples, which are intended for illustrative purposes and are not intended to limit the scope of the disclosure or its application in any way.

### Example 1. Synthesis of (Z)-2-(heptadec-8-en-1-yl)-1-(2-hydroxyethyl)-3-(2-hydroxypropyl)-4,5-dihydro-1H-imidazol-3-ium Gluconate (Formula 1)

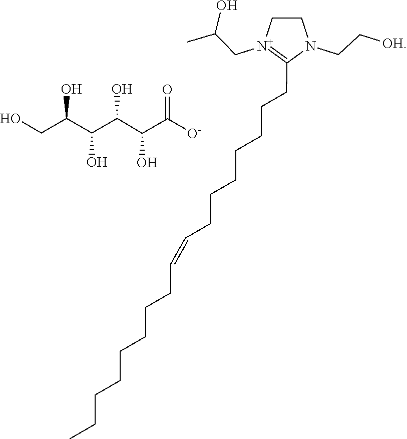 [SMILES: O=C([O-])[C@H](O)[C@@H](O)[C@H](O)[C@H](O)CO ; CCCCCCCC/C=C\CCCCCCCC1=[N+](CC(C)O)CCN1CCO]

Into a 1-L three-necked round-bottomed flask, 173.369 g (494.5 mmoles) of (Z)-2-(2-(heptadec-8-en-1-yl)-4,5-dihydro-1H-imidazol-1-yl)-ethan-1-ol was charged. The (Z)-2-(2-(heptadec-8-en-1-yl)-4,5-dihydro-1H-imidazol-1-yl)-ethan-1-ol was prepared according to methods commonly known to those of ordinary skill in the art.

A magnetic follower and 100 g of methanol and 100 g of deionized water were charged into the flask. The flask was placed into a heating mantle with a magnetic stirrer. The mixture was stirred until it was homogenous. Gluconic acid (195.013 g; 495.4 mmol.), was slowly added into the flask under vigorous stirring. Thereafter, the flask was armed with a dropping funnel, thermocouple and a water-cooled condenser. Propylene oxide (29.012 g; 495.4 mmol), was added into the dropping funnel. The reaction mixture was heated under reflux at 50° C. as propylene oxide was added dropwise into the reaction mixture. On the complete transfer of the propylene oxide, the reaction was left to ride for 60 minutes. It was cooled to 25° C. to provide Formula 1.

### Example 2. Synthesis of (Z)-2-(heptadec-8-en-1-yl)-3-(2-hydroxy-2-phenylethyl)-1-(2-hydroxyethyl)-4,5-dihydro-1H-imidazol-3-ium Gluconate (Formula 2)

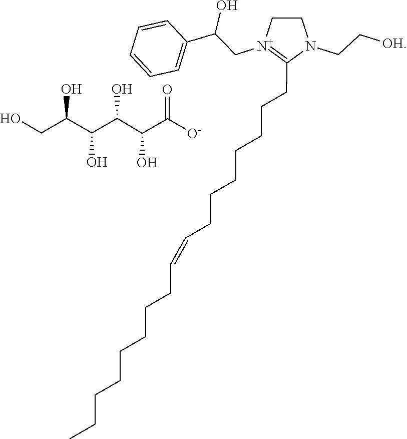 [SMILES: O=C([O-])[C@H](O)[C@@H](O)[C@H](O)[C@H](O)CO ; CCCCCCCC/C=C\CCCCCCCC1=[N+](CC(O)c2ccccc2)CCN1CCO]

The procedure in Example 1 was used to synthesize (Z)-2-(heptadec-8-en-1-yl)-3-(2-hydroxy-2-phenylethyl)-1-(2-hydroxyethyl)-4,5-dihydro-1H-imidazol-3-ium gluconate as Formula 2.

### Example 3. Blank Wheel Box Test

To determine the efficacy of the compositions as corrosion inhibitors, a wheel box test was used. It is a testing methodology from NACE publication ID182. The method is used to assess the degree of protection afforded to a metal surface that is in contact with water laden with carbon dioxide (CO₂). The wheel box test is a test that is often used in the field for comparative corrosion inhibitor performance tests. The standard test conditions used in the performance test are in Table 1.

TABLE 1
Wheel Box Test Conditions

| Temperature | 80° | C. |
|---|---|---|
| Oil type | 20% LVT-200 | |
| Brine¹ | 80% ASTM Seawater brine | |
| CO₂ level | Saturated | |
| Duration | 24 | hours |

¹The brine solution consisted of 0.47 g/L of CaCl₂•2H₂O, 0.60 g/L of MgCl₂—6H₂0, 0.11 g/L of KCl, 0.08 g/L of Na₂SO₄, and 1.4 g/L of NaHCO₃.

A continuous wheel box test procedure was used to compare the performance of the corrosion inhibitor formulations of the invention in mitigating the corrosion of carbon steel in solutions saturated with carbon dioxide. Into a set of bottles containing the desired brine and oil mixtures, Table 1, clean and pre-weighed coupons were immersed. The solution was purged with carbon dioxide, after which the solutions were dosed with the various concentrations of the formulations. After purging the headspace with carbon dioxide, the bottles were immediately capped to prevent aerial exposure.

Blanks, comprising bottles of untreated mixtures, were used to determine the baseline corrosion rate under the test conditions. The bottles were placed on a rotating wheel under incubation at 80° C. for a period of 24 hours. Following the completion of the incubation, the bottles were retrieved and left to cooled to room temperature. The coupons were removed from the test solutions, cleaned, dried, sandblasted and re-weighed. By comparing the mass loss of the coupons immersed in treated solutions versus the mass loss for coupons placed in untreated solutions, the corrosion rate and protection efficiency were calculated.

TABLE 2
Blank Corrosion Test (in triplicate)

| | Inhibitor Conc. | Weight | | % |
|---|---|---|---|---|
| | (ppm) | Loss Mg. | MPY | Protection |
| BLANK | 0 | 67.7 | 41.30 | 0% |
| | 0 | 68.8 | 41.97 | 0% |
| | 0 | 65.1 | 39.71 | 0% |

The data in Table 2 demonstrate that the test coupons were not protected in the presence of untreated test solutions.

### Example 4. Comparative Formulation Wheel Box Test

For comparative purposes, 676 mmolal solution of (Z)-2-(heptadec-8-en-1-yl)-1-(2-hydroxyethyl)-4,5-dihydro-1H-imidazol-3-ium gluconate was prepared using (Z)-2-(2-(heptadec-8-en-1-yl)-4,5-dihydro-1H-imidazol-1-yl)ethan-1-ol and gluconic acid. This formulation was designated as the Comparative Composition.

### Example 5. (Z)-2-(Heptadec-8-en-1-yl)-1-(2-hydroxyethyl)-3-(2-hydroxypropyl)-4,5-dihydro-1H-imidazol-3-ium Gluconate Wheel Box Test

For determination of the protective efficacy of the prototype represented by novel architecture (Z)-2-(heptadec-8-en-1-yl)-1-(2-hydroxyethyl)-3-(2-hydroxypropyl)-4,5-dihydro-1H-imidazol-3-ium gluconate prepared in Example 1, composition in said example was diluted to 674.6 mmolal. The formulation was denoted as Novel Composition 1. The corrosion protection imparted by this formulation was assessed using the procedure in Example 3.

According to the test, the novel (Z)-2-(heptadec-8-en-1-yl)-1-(2-hydroxyethyl)-3-(2-hydroxypropyl)-4,5-dihydro-1H-imidazol-3-ium gluconate displayed a surprisingly high efficacy in terms of corrosion protection, Table 3.

### Example 6. (Z)-2-(Heptadec-8-en-1-yl)-1-(2-hydroxyethyl)-3-(2-hydroxy-2-phenylethyl)-4,5-dihydro-1H-imidazol-3-ium Gluconate Wheel Box Test

Similarly, a composition of the prototype of the architecture, a 674.6 mmolal solution of (Z)-2-(heptadec-8-en-1-yl)-1-(2-hydroxyethyl)-3-(2-hydroxy-2-phenylethyl)-4,5-dihydro-1H-imidazol-3-ium gluconate was prepared using the product in Example 2. This was Novel Composition 2. To determine the effectiveness of the corrosion protection imparted by this formulation was assessed using the procedure in Example 3.

TABLE 3
Performance of current gluconate salt of non-derivatized imidazoline,
gluconate salt of imidazoline quaternarized with propylene oxide,
and gluconate salt of imidazoline quaternarized with styrene oxide.

| Inhibitor | | 10 ppm | 25 ppm | 50 ppm |
|---|---|---|---|---|
| Blank | % Protection | 0 | 0 | 0 |
| Control | % Protection | 26.5 | 43.5 | 50.0 |
| Composition 1 | % Protection | 33.0 | 63.0 | 73.0 |
| | % Improvement | 24 | 45 | 46 |
| Composition 2 | % Protection | 45.0 | 65.0 | 79.5 |
| | % Improvement | 70 | 49 | 59 |

The results showed that both (Z)-2-(heptadec-8-en-1-yl)-1-(2-hydroxyethyl)-3-(2-hydroxypropyl)-4,5-dihydro-1H-imidazol-3-ium gluconate and (Z)-2-(heptadec-8-en-1-yl)-1-(2-hydroxyethyl)-3-(2-hydroxy-2-phenylethyl)-4,5-dihydro-1H-imidazol-3-ium gluconate displayed surprisingly excellent corrosion protection efficiency even at low concentrations, Table 3.

All of the compositions and methods disclosed and claimed herein can be made and executed without undue experimentation in light of the present disclosure. While this invention may be embodied in many different forms, there are described in detail herein specific preferred embodiments of the invention. The present disclosure is an exemplification of the principles of the invention and is not intended to limit the invention to the particular embodiments illustrated. In addition, unless expressly stated to the contrary, use of the term “a” is intended to include “at least one” or “one or more.” For example, “a corrosion inhibitor” is intended to include “at least one corrosion inhibitor” or “one or more corrosion inhibitors.”

Any ranges given either in absolute terms or in approximate terms are intended to encompass both, and any definitions used herein are intended to be clarifying and not limiting. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges (including all fractional and whole values) subsumed therein.

Any composition disclosed herein may comprise, consist of, or consist essentially of any element, component and/or ingredient disclosed herein or any combination of two or more of the elements, components or ingredients disclosed herein.

Any method disclosed herein may comprise, consist of, or consist essentially of any method step disclosed herein or any combination of two or more of the method steps disclosed herein.

The transitional phrase “comprising,” which is synonymous with “including,” “containing,” or “characterized by,” is inclusive or open-ended and does not exclude additional, un-recited elements, components, ingredients and/or method steps.

The transitional phrase “consisting of” excludes any element, component, ingredient, and/or method step not specified in the claim.

The transitional phrase “consisting essentially of” limits the scope of a claim to the specified elements, components, ingredients and/or steps, as well as those that do not materially affect the basic and novel characteristic(s) of the claimed invention.

Unless specified otherwise, all molecular weights referred to herein are weight average molecular weights and all viscosities were measured at 25° C. with neat (not diluted) polymers.

As used herein, the term “about” refers to the cited value being within the errors arising from the standard deviation found in their respective testing measurements, and if those errors cannot be determined, then “about” may refer to, for example, within 5%, 4%, 3%, 2%, or 1% of the cited value.

Furthermore, the invention encompasses any and all possible combinations of some or all of the various embodiments described herein. It should also be understood that various changes and modifications to the presently preferred embodiments described herein will be apparent to those skilled in the art. Such changes and modifications can be made without departing from the spirit and scope of the invention and without diminishing its intended advantages. It is therefore intended that such changes and modifications be covered by the appended claims.

What is claimed is:

1. A corrosion inhibitor composition comprising a compound of formula (I): 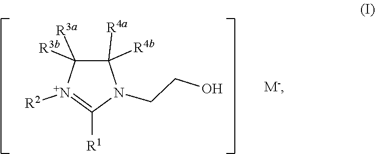
wherein:
R¹ is an optionally substituted C₈-C₂₀ alkyl functional group, an optionally substituted C₈-C₂₀ alkenyl with one or more vinylic moieties, or an optionally substituted C₈-C₂₀ alkynyl with one or more vinylic moieties, an optionally substituted —(C₆-C₂₀ aryl)-(C₄-C₂₀ alkyl), an optionally substituted
—(C₄-C₂₀ alkylene)-(C₆-C₂₀ aryl), or an optionally substituted
—(C₄-C₂₀ alkylene)-(C₆-C₂₀ aryl)-(C₄-C₂₀ alkyl);
R² is optionally substituted C₁-C₂₀ alkyl, optionally substituted
C₂-C₂₀ β-hydroxyalkyl, optionally substituted —(C₁-C₂₀ alkylene)-(C₆-C₂₀ aryl), optionally substituted —(C₂-C₂₀ hydroxyalkylene)-(C₆-C₂₀ aryl), or optionally substituted —(C₂-C₂₀ hydroxyalkylene)-(C₆-C₂₀ aryl)-(C₆-C₂₀ alkyl);
R^{3a} is H, —OH, —NH₂, —NH(C₁-C₆ alkyl), —N(C₁-C₆ alkyl)₂, —CN, halo, or optionally substituted C₁-C₆ alkyl;
R^{3b} is H, —OH, —NH₂, —NH(C₁-C₆ alkyl), —N(C₁-C₆ alkyl)₂, —CN, halo, or optionally substituted C₁-C₆ alkyl;
R^{4a} is H, —OH, —NH₂, —NH(C₁-C₆ alkyl), —N(C₁-C₆ alkyl)₂, —CN, halo, or optionally substituted C₁-C₆ alkyl;
R^{4b} is H, —OH, —NH₂, —NH(C₁-C₆ alkyl), —N(C₁-C₆ alkyl)₂, —CN, halo, or optionally substituted C₁-C₆ alkyl; and
M⁻ is an inorganic, organic counter ion or a combination of both.

2. The composition of claim 1, wherein R² is optionally substituted C₂-C₂₀ β-hydroxyalkyl or optionally substituted —(C₁-C₆ hydroxyalkylene)-(phenyl).

3. The composition of claim 1, wherein R² is —CH₂CH₂(OH), —CH₂CH(OH)CH₃, or —CH₂CH(OH)-(phenyl).

4. The composition of claim 1, wherein the compound of formula (I) is of formula (I-A): 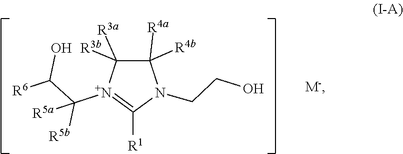
wherein:
R¹ is an optionally substituted C₈-C₂₀ alkyl, an optionally substituted
C₈-C₂₀ alkenyl with one or more vinylic moieties, or an optionally substituted C₈-C₂₀ alkynyl with one or more vinylic moieties;
R^{3a} is H, —OH, —NH₂, —NH(C₁-C₆ alkyl), —N(C₁-C₆ alkyl)₂, —CN, halo, or optionally substituted C₁-C₆ alkyl;
R^{3b} is H, —OH, —NH₂, —NH(C₁-C₆ alkyl), —N(C₁-C₆ alkyl)₂, —CN, halo, or optionally substituted C₁-C₆ alkyl;
R^{4a} is H, —OH, —NH₂, —NH(C₁-C₆ alkyl), —N(C₁-C₆ alkyl)₂, —CN, halo, or optionally substituted C₁-C₆ alkyl;
R^{4b} is H, —OH, —NH₂, —NH(C₁-C₆ alkyl), —N(C₁-C₆ alkyl)₂, —CN, halo, or optionally substituted C₁-C₆ alkyl;
R^{5a} is H, —OH, —NH₂, —NH(C₁-C₆ alkyl), —N(C₁-C₆ alkyl)₂, —CN, halo, or optionally substituted C₁-C₆ alkyl;
R^{5b} is H, —OH, —NH₂, —NH(C₁-C₆ alkyl), —N(C₁-C₆ alkyl)₂, —CN, halo, or optionally substituted C₁-C₆ alkyl;
R⁶ is optionally H, substituted C₁-C₂₀ alkyl, optionally substituted
C₁-C₂₀ aryl, or optionally substituted —(C₆-C₂₀ aryl)-(C₄-C₂₀ alkyl); and
M⁻ is a counter ion.

5. The composition of claim 1, wherein R¹ is selected from octyl, octenyl, octynyl, nonyl, nonenyl, nonynyl, decyl, decenyl, decynyl, undecyl, undecenyl, undecynyl, dodecyl, dodecenyl, dodecynyl, tridecyl, tridecenyl, tridecynyl, tetradecyl, tetradecenyl, tetradecynyl, pentadecyl, pentadecenyl, pentadecynyl, hexadecyl, hexadecenyl, hexadecynyl, heptadecyl, heptadecenyl, heptadecynyl, octadecyl, octadecenyl, octadecynyl, nonadecyl, nonadecenyl, nonadecynyl, icosyl, icosenyl, and icosynyl.

6. The composition of claim 1, wherein R^{3a} is H, wherein R^{3b} is H, wherein R^{4a} is H, and/or wherein R^{4b} is H.

7. The composition of claim 1, wherein M⁻ is selected from sulfate, phosphate, nitrate, sulfonate, iodide, fluoride, chloride, phosphate, phenolates, gluconate, allarate, altarate, altrarate, altronate, arabinarate, arabinonate, citrate, dihomocitrate, fructuronate, fuconate, fumarate, galactarate, galactonate, galacturonate, glucarate, glucoheptonate, gluconate, glucuronate, gulonate, homocitrate, homoisocitrate, idarate, idonate, iduronate, isocitrate, mannarate, mannonate, lactate, malate, tartarate, octulosonate, rhamnonate, ribonate, tagaturonate, xylonate, xyluronate, tartarate, tatronate, glycerate, malonate, pantoate, or a combination thereof.

8. The composition of claim 1, wherein the compound of formula (I) is selected from: 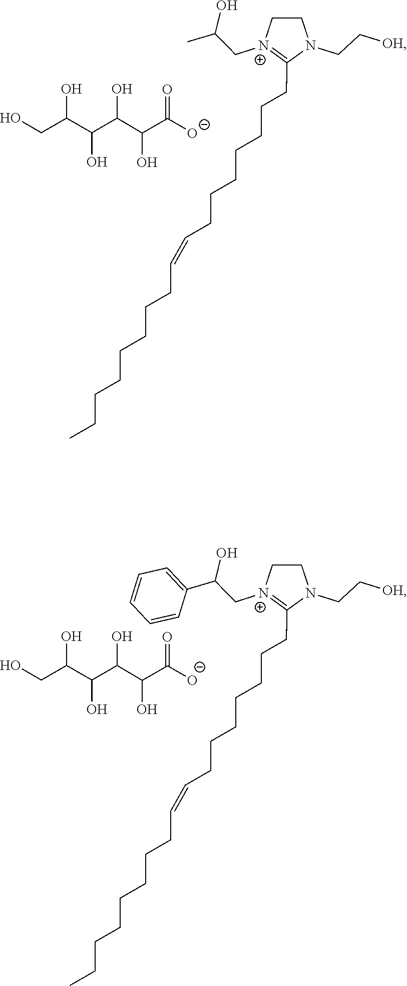 [SMILES: O=C([O-])C(O)C(O)C(O)C(O)CO ; CCCCCCCC/C=C\CCCCCCCC1=[N+](CC(C)O)CCN1CCO ; O=C([O-])C(O)C(O)C(O)C(O)CO ; CCCCCCCC/C=C\CCCCCCCC1=[N+](CC(O)c2ccccc2)CCN1CCO]
and combinations thereof.

9. The composition of claim 1, further comprising an antiscalant, a hydrate inhibitor, a phosphate ester, a mercaptan, a molybdophospate, or any combination thereof.

10. The composition of claim 9, wherein the antiscalant is selected from allaric acid, altaric acid, altraric acid, altronic acid, arabinaric acid, arabinonic acid, citric acid, dihomocitric acid, fructuronic acid, fuconic acid, fumaric acid, galactaric acid, galactonic acid, galacturonic acid, glucaric acid, glucoheptonic acid, gluconic acid, glucuronic acid, gulonic acid, homocitric acid, homoisocitric acid, idaric acid, idonic acid, iduronic acid, isocitric acid, mannaric acid, mannonic acid, malic acid, tartaric acid, octulosonic acid, rhamnonic acid, ribonic acid, tagaturonic acid, xylonic acid, xyluronic acid, tartaric acid, tatronic acid, glyceric acid, malonic acid, and pantoic acid, or a salt thereof.

11. A method of reducing corrosion of a metal surface in contact with an aqueous system, comprising adding the corrosion inhibitor composition of claim 1 to the aqueous system.

12. The method of claim 11, wherein the aqueous system comprises a corrosive agent.

13. The method of claim 12, wherein the corrosive agent is selected from a brine, an acid, carbon dioxide, hydrogen sulfide, or a combination thereof.

14. The method of claim 11, wherein the metal surface comprises metallic-chrome steel, ferritic-alloy steel, austenitic-steel, precipitation-hardened steel, high-nickel steel, carbon steel, or a combination thereof.

15. A process for preparing a compound of formula (I-A): 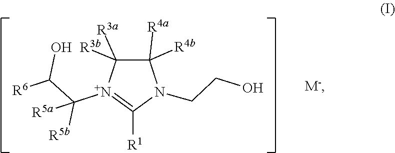
comprising contacting a compound of formula (I-B): 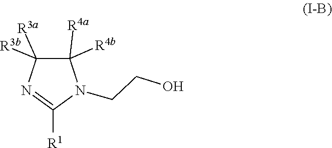
with a compound of formula (I-C): 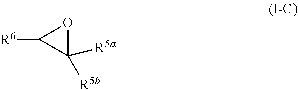
wherein:
R¹ is optionally substituted C₈-C₂₀ alkyl, an optionally substituted
C₈-C₂₀ alkenyl with one or more vinylic moieties, or an optionally substituted C₈-C₂₀ alkynyl with one or more vinylic moieties, an optionally substituted —(C₆-C₂₀ aryl)-(C₄-C₂₀ alkyl), an optionally substituted
—(C₄-C₂₀ alkylene)-(C₆-C₂₀ aryl), or an optionally substituted
—(C₄-C₂₀ alkylene)-(C₆-C₂₀ aryl)-(C₄-C₂₀ alkyl);
R^{3a} is H, —OH, —NH₂, —NH(C₁-C₆ alkyl), —N(C₁-C₆ alkyl)₂, —CN, halo, or optionally substituted C₁-C₆ alkyl;
R^{3b} is H, —OH, —NH₂, —NH(C₁-C₆ alkyl), —N(C₁-C₆ alkyl)₂, —CN, halo, or optionally substituted C₁-C₆ alkyl;
R^{4a} is H, —OH, —NH₂, —NH(C₁-C₆ alkyl), —N(C₁-C₆ alkyl)₂, —CN, halo, or optionally substituted C₁-C₆ alkyl;
R^{4b} is H, —OH, —NH₂, —NH(C₁-C₆ alkyl), —N(C₁-C₆ alkyl)₂, —CN, halo, or optionally substituted C₁-C₆ alkyl; and
R^{5a} is H, —OH, —NH₂, —NH(C₁-C₆ alkyl), —N(C₁-C₆ alkyl)₂, —CN, halo, or optionally substituted C₁-C₆ alkyl;
R^{5b} is H, —OH, —NH₂, —NH(C₁-C₆ alkyl), —N(C₁-C₆ alkyl)₂, —CN, halo, or optionally substituted C₁-C₆ alkyl;
R⁶ is optionally substituted C₁-C₆ alkyl or optionally substituted C₆-C₁₀ aryl; and
M⁻ is a counter ion.

16. The process of claim 15, wherein contacting a compound of formula (I-B) with a compound of formula (I-C) is performed in the presence of an acid.

17. The process of claim 16, wherein the acid is selected from gluconic acid, allaric acid, altaric acid, altraric acid, altronic acid, arabinaric acid, arabinonic acid, citric acid, dihomocitric acid, fructuronic acid, fuconic acid, fumaric acid, galactaric acid, galactonic acid, galacturonic acid, glucaric acid, glucoheptonic acid, gluconic acid, glucuronic acid, gulonic acid, homocitric acid, homoisocitric acid, idaric acid, idonic acid, iduronic acid, isocitric acid, mannaric acid, mannonic acid, malic acid, tartaric acid, octulosonic acid, rhamnonic acid, ribonic acid, tagaturonic acid, xylonic acid, xyluronic acid, tartaric acid, tatronic acid, glyceric acid, malonic acid, lactic acid, acetic acid, propionic acid, butyric acid, pentanoic acid, hexanoic acid, heptanoic acid, octanoic acid, nonanoic acid, decanoic acid, undecanoic acid, dodecanoic acid, tetradecanoic acid, pentadecanoic acid, hexadecenoic acid, septadecanoic acid, octadecanoic acid, pantoic acid, hydrochloric acid, sulfuric acid, sulfonic acid, phosphoric acid, nitric acid, iodic acid, bromic acid, or a combination thereof.

18. The process of claim 15, wherein contacting a compound of formula (I-B) with a compound of formula (I-C) is performed in the presence of heat.

19. The process of claim 15, further comprising contacting a compound of formula (I-D): 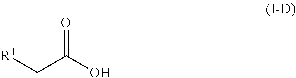
with a compound of formula (I-E): 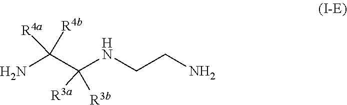
to produce the compound of formula (I-B).

20. The process of claim 15, wherein M⁻ is selected from sulfate, phosphate, nitrate, sulfonate, iodide, fluoride, chloride, gluconate, allarate, altarate, altrarate, altronate, arabinarate, arabinonate, citrate, dihomocitrate, fructuronate, fuconate, fumarate, galactarate, galactonate, galacturonate, glucarate, glucoheptonate, gluconate, glucuronate, gulonate, homocitrate, homoisocitrate, idarate, idonate, iduronate, isocitrate, mannarate, mannonate, malate, tartarate, octulosonate, rhamnonate, ribonate, tagaturonate, xylonate, xyluronate, tartarate, tatronate, glycerate, malonate, pantoate, lactate or a combination thereof.
